(19) United States
(12) Patent Application Publication
(10) Pub. No.: US 2024/0115729 A1
(43) Pub. Date: Apr. 11, 2024

(54) COMPOSITIONS AND METHODS FOR TARGETED ANTIFIBROTIC THERAPY IN CHRONIC PANCREATITIS

(71) Applicant: University of Virginia Patent Foundation, Charlottesville, VA (US)

(72) Inventors: Kimberly A. Kelly, Ph.D., Goochland, VA (US); Jessica Hung, Charlottesville, VA (US)

(73) Assignee: University of Virginia Patent Foundation, Charlottesville, VA (US)

(21) Appl. No.: 18/276,111

(22) PCT Filed: Feb. 4, 2022
(86) PCT No.: PCT/US2022/015272

§ 371 Date: Aug. 7, 2023

Related U.S. Application Data
(60) Provisional application No. 63/146,401, filed on Feb. 5, 2021.

(51) Int. Cl. A61K 47/69; A61K 31/352; A61K 45/06; A61K 47/58; A61P 5/00; C07K 7/06

(52) U.S. Cl. CPC A61K 47/6911; A61K 31/352; A61K 45/06; A61K 47/605; A61P 5/00; C07K 7/06

(57) ABSTRACT

Provided are compositions that include targeting peptides and methods for using the same to treat and/or prevent various diseases, disorders, and/or conditions. In some embodiments, the compositions and methods relate to liposomal compositions that include a liposome, the surface of which is conjugated to a peptide having an amino acid sequence as set forth in any of SEQ ID NOS: 3-38, optionally wherein the liposome encapsulates a therapeutic agent or a detectable agent. In some embodiments, the peptide has an amino acid sequence that is one of SEQ ID NOs: 14, 19, 20, 27, and 28. Also provided are methods treating or preventing fibrosis, for decreasing the incidence of a disease, disorder, or condition associated with chronic pancreatitis (CP), for targeting active agents to targets, including but not limited to collagen III-expressing cells and extracellular matrix, and for decreasing incidence of side effects associated with apigenin treatment.

### CROSS REFERENCE TO RELATED APPLICATION

The presently disclosed subject matter claims the benefit of U.S. Provisional Patent Application Ser. No. 63/146,401, filed Feb. 5, 2021, the disclosure of which incorporated herein by reference in its entirety.

### GOVERNMENT INTEREST

This invention was made with government support under Grant Nos. CA044579, CA231196, and CA233716 awarded by The National Institutes of Health. The government has certain rights in the invention.

### REFERENCE TO SEQUENCE LISTING SUBMITTED ELECTRONICALLY

The content of the electronically submitted sequence listing in ASCII text file (Name: 3062 146 PCT_ST25.txt; Size: 17 kilobytes; and Date of Creation: Feb. 4, 2022) filed with the instant application is incorporated herein by reference in its entirety.

### TECHNICAL FIELD

The presently disclosed subject matter relates in some embodiments to compositions that target sites of fibrosis and methods of using the same to prevent and/or treat fibrosis and diseases, disorders, and conditions associated therewith. In particular embodiments, the presently disclosed subject matter provides compositions, optionally liposomal compositions, that comprise peptides that target the compositions to collagen III⁺-expressing cells to deliver therapeutically active molecules thereto.

### BACKGROUND

Chronic pancreatitis (CP) is an inflammatory disorder that causes irreversible damage in the pancreas and induces long-standing sequelae encompassing recurrent severe pain, fibrosis, duct distortion, parenchymal calcification, and loss of exocrine and endocrine function at the advanced stage (Beyer et al., 2020; Gardner et al., 2020). In addition to physical debilities, patients with CP typically struggle with psychological and financial challenges, resulting in a significantly impaired quality of life (Morgan et al., 2012;

Machicado et al., 2017; Han et al., 2018). Patients with a history of CP have an increased risk of developing pulmonary diseases, diabetes mellitus, and pancreatic cancer (Bang et al., 2014). The annual incidence of CP worldwide ranges between 4.4-14 per 100,000 population, with an approximate prevalence of 36.9-52.4 per 100,000 (Kichler & Jang, 2020). Despite low prevalence, the frequent demands of pain management and necessary procedures directly or indirectly caused by CP lead to a disproportional high cost of medical care, resulting in a high socioeconomic burden on diseased individuals and the health care system in the United States and throughout the world (Hall et al., 2014; Ting et al., 2016; Machicado et al., 2019).

Fibrosis is a characteristic feature of CP, which is not only an outcome of recurrent pancreatic parenchymal cell necrosis but is also responsible for the post-injury reactions that induce a cascade of events and signifies molecular and cellular damage. Various cellular components and molecular crosstalk are involved in fibrogenesis, with each of the components contributing to the clinical outcome of pancreatic remodeling. However, no FDA-approved drug is available to address the fundamental causes of inflammation and fibrogenesis and to halt and reverse the damage of pancreatitis. In fact, current therapeutic strategies for CP are limited to palliative care and pain alleviation and these approaches fail at the advanced stage when invasive surgical procedures are the only available options.

Thus, addressing fibrotic conditions has become one of the main areas to improve pancreatitis outcomes. Apigenin, a natural, small molecule compound, has been shown preclinically to have antifibrotic, anti-inflammatory, antioxidant, and proapoptotic properties in CP and various cancers in vitro and in vivo (Mrazek et al., 2015; Mrazek et al., 2019; Ahmed et al., 2021). Despite these promising features, apigenin suffers from low aqueous solubility, metabolic instability, and off-target effects (Chen et al., 2014); therefore, no approved clinical applications of apigenin are available.

### SUMMARY

This Summary lists several embodiments of the presently disclosed subject matter, and in many cases lists variations and permutations of these embodiments of the presently disclosed subject matter. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

In some embodiments, the presently disclosed subject matter relates to liposomal compositions comprising a liposome, wherein a surface of the liposome has been conjugated to a peptide selected from the group consisting of SEQ ID NOs: 3-38, optionally wherein the liposome encapsulates an active agent selected from the group consisting of a therapeutic agent and a detectable agent. In some embodiments, the peptide is selected from the group consisting of KTYVPTT (SEQ ID NO: 14), MDLSLKP (SEQ ID NO: 19), MNSIAIP (SEQ ID NO: 20) , SLTNSSF (SEQ ID NO: 27), and SNSQDLH (SEQ ID NO: 28). In some embodiments, the peptide is MDLSLKP (SEQ ID NO: 19) and the liposome is targeted to a cell expressing collagen IIIa. In some embodiments, the therapeutic agent is an antifibrotic agent, optionally apigenin. In some embodiments, a liposome composition of the presently disclosed subject matter further comprises a pharmaceutically acceptable carrier, diluent, and/or excipient, optionally, wherein the pharmaceutically acceptable carrier, diluent, and/or excipient is pharmaceutically acceptable for use in a subject.

In some embodiments, the presently disclosed subject matter also relates to peptides comprising, consisting essentially of, or consisting of an amino acid sequence as set forth in any one of SEQ ID NOs: 3-38 or a mimetic, analog, or derivative thereof, optionally wherein the amino acid sequence is no more than 10 amino acids, 20 amino acids, 30 amino acids, 40 amino acids, or 50 amino acids. In some embodiments, one or more of the amino acids in the amino acid sequence is a modified amino acid and/or a non-standard amino acid, further optionally wherein the amino acid sequence as set forth in any one of SEQ ID NOs: 3-38 is unmodified. In some embodiments, the peptide is selected from the group consisting of KTYVPTT (SEQ ID NO: 14), MDLSLKP (SEQ ID NO: 19), MNSIAIP (SEQ ID NO: 20), SLTNSSF (SEQ ID NO: 27), and SNSQDLH (SEQ ID NO: 28).

In some embodiments, the presently disclosed subject matter also relates to methods for treating and/or preventing fibrosis. In some embodiments, the methods comprise administering to a subject in need thereof a therapeutically effective amount of a liposomal composition as disclosed herein. In some embodiments, the peptide is MDLSLKP (SEQ ID NO: 19) and the liposome targets a cell expressing collagen IIIa that is present in the subject. In some embodiments, the cell is present in the pancreas of the subject and the subject is at risk for developing chronic pancreatitis (CP) and/or pancreatic fibrosis

In some embodiments, the presently disclosed subject matter also relates to methods for decreasing the incidence of diseases, disorders, and/or conditions associated with chronic pancreatitis (CP). In some embodiments, the methods comprise administering to a subject in need thereof a therapeutically effective amount of a liposomal composition as disclosed herein. In some embodiments, the peptide is MDLSLKP (SEQ ID NO: 19) and the liposome targets a cell expressing collagen IIIa that is present in the subject. In some embodiments, the disease, disorder, and/or condition associated with CP is selected from the group consisting of pulmonary disease, diabetes mellitus, and pancreatic cancer.

In some embodiments, the presently disclosed subject matter also relates to methods for targeting active agents to targets. In some embodiments, the methods comprise contacting the target with a liposomal composition as disclosed herein. In some embodiments, the target is selected from the group consisting of an acinar cell, an activated pancreatic stellate cell (aPSC), a component of the extracellular matrix (ECM), and a macrophage. In some embodiments, the liposomal composition comprises a peptide comprising, consisting essentially of, or consisting of an amino acid sequence as set forth in any one of SEQ ID NOs: 14, 19, 20, 27, and 28. In some embodiments, the active agent is a therapeutic agent, optionally an antifibrotic agent and/or a chemotherapeutic agent, or a detectable agent.

In some embodiments, the presently disclosed subject matter also relates to methods for decreasing incidence of side effects associated with apigenin treatment in subjects. In some embodiments, the methods comprise administering to the subject a liposome, wherein a surface of the liposome has been conjugated to a peptide selected from the group consisting of SEQ ID NOs: 3-38, and further wherein the liposome encapsulates the apigenin. In some embodiments, the side effect associated with apigenin treatment comprises hepatotoxicity. In some embodiments, the subject has or is at risk of developing chronic pancreatitis (CP) and/or a disease, disorder, and/or condition associated with CP.

In some embodiments, the presently disclosed subject matter also relates to methods for targeting active agents to collagen III-expressing cells. In some embodiments, the methods comprise contacting a collagen III-expressing cell with a vehicle comprising the active agent and a peptide comprising, consisting essentially of, or consisting of the amino acid sequence MDLSLKP (SEQ ID NO: 19), wherein the peptide binds to collagen III on or in the cell to thereby target the active agent to the collagen III-expressing cell. In some embodiments, the collagen III is present in the extracellular matrix of the cell. In some embodiments, the cell is present in a subject, optionally in the pancreas of the subject. In some embodiments, the cell is present in the pancreas of the subject. In some embodiments, the subject has or is at risk of developing chronic pancreatitis (CP) and/or a disease, disorder, and/or condition associated with CP.

In some embodiments, the presently disclosed subject matter also relates to methods for delivering active agents to targets, optionally target in subjects, the methods comprising contacting the target with a vehicle comprising the active agent and a peptide comprising, consisting essentially of, or consisting of an amino acid sequence as set forth in any of SEQ ID NOs: 14, 19, 20, 27, and 28, wherein the peptide binds to the target to thereby deliver the active agent to the target. In some embodiments, the target is selected from the group consisting of (i) a collagen III antigen, optionally a collagen III antigen that is present in the extracellular matrix of a cell, and the peptide comprises, consists essentially of, or consists of SEQ ID NO. 19; (ii) a carboxypeptidase A1 (CPA-1) antigen, optionally a CPA-1 antigen that is present on a pancreatic acinar cell, and the peptide comprises, consists essentially of, or consists of SEQ ID NO: 20; (iii) an α-smooth muscle actin (α-SMA) antigen, optionally an α-SMA antigen that is present on a cell, and the peptide comprises, consists essentially of, or consists of SEQ ID NO: 14; and (iv) an F4/80 antigen, optionally an F4/80 antigen that is present on a cell, and the peptide comprises, consists essentially of, or consists of SEQ ID NO: 27 and/or SEQ ID NO: 28. In some embodiments, the vehicle comprises a plurality of peptides comprising, consisting essentially of, or consisting of at least two amino acid sequence selected from the group consisting of SEQ ID NOs: 14, 19, 20, 27, and 28. In some embodiments, the target is a cell present in a subject, optionally in the pancreas of the subject. In some embodiments, the cell is present in the pancreas of the subject. In some embodiments, the subject bas or is at risk of developing chronic pancreatitis (CP) and/or a disease, disorder, and/or condition associated with CP.

In some embodiments, the presently disclosed subject matter also relates to methods for delivering active agents to extracellular matrix (ECM), optionally ECM present in a subject. In some embodiments, the methods comprise contacting the ECM with a vehicle comprising the active agent and a peptide comprising, consisting essentially of, or consisting of an amino acid sequence as set forth in SEQ ID NO: 19, wherein the peptide binds to the ECM to thereby deliver the active agent to the target. In some embodiments, the ECM is associated with fibrosis in the subject, and the active agent treats or prevents the development and/or progression of the fibrosis in the subject. In some embodiments, the fibrosis in the subject is associated with chronic pancreatitis (CP) and/or a disease, disorder, and/or condition associated with CP, pancreatic cancer, pulmonary disease, diabetes mellitus, hepatic fibrosis, or any combination thereof.

Thus, it is an object of the presently disclosed subject matter to provide a systematic approach to profile peptide ligands specific for cellular components of complex disease models, and demonstrates the biomedical applications of the identified peptides to improve tissue remodeling in the inflamed pancreas.

An object of the presently disclosed subject matter having been stated herein above, and which is achieved in whole or in part by the presently disclosed subject matter, other objects will become evident as the description proceeds when taken in connection with the accompanying Figures as best described herein below.

### BRIEF DESCRIPTION OF THE FIGURES

FIGS. 1A and 1B. Caerulein-induced CP mouse model. (FIG. 1A) Caerulein injection schedule (FIG. 1B) Pancreas H&E and Picrosirius red staining of healthy C57BL/6J and caerulein-induced CP mice. Acinar cell atrophy (black arrows) and fibroblast/immune cell infiltrations (white arrows) are observed in CP mice. Brightfield 50×.

FIG. 2. Groupings and validations of the selected CP-specific clones. Candidate clones grouping based on sequence similarity by GibbsCluster Server 2.0. for group validation.

FIGS. 3A-3C. The size distribution of peptide modified liposomes detected by NanoSight. (FIG. 3A) NP liposomes (FIG. 3B) MDLSLKP (SEQ ID NO: 19) liposomes. (FIG. 3C) MNSIAIP (SEQ ID NO: 20) liposomes.

FIGS. 4A and 4B. Fibronectin staining in tissues other than pancreas. (FIG. 4A) Images of liver, heart, and lung stained with fibronectin harvested from mice treated with vehicle, free apigenin, apigenin-encapsulated control liposomes, and apigenin-encapsulated ECM liposomes. (FIG. 4B) Quantification of percent fibronectin-positive area in liver, heart, and lung. n=4 images/group. ANOVA was used: p=0.699 (liver), 0.777 (heart), and 0.074 (lung).

FIGS. 5A and 5B. In vivo phage display in chronic pancreatitis. (FIG. 5A) Schematic of an exemplary in vivo phage biopanning process to screen for clones specific for CP pancreas. (FIG. 5B) Phage titering of in vivo phage screening in caerulein-induced CP mice. Phage pools (% injected dose per gram tissue) were recovered from the pancreas and various organs in 3 rounds of the biopanning process. One-way ANOVA and Tukey-Kramer tests were used to compare round 3 vs. round 1, and round 3 vs. round 2. n=3; ***p<0.0001. SK M: Skeletal muscle.

FIGS. 6A-6F. In silico selection and in vivo validation of CP targeting candidate clones. CP targeting candidate clones were selected based on PHASTpep (FIG. 6A), replicates between 3 mice (FIG. 6B), clone enrichment between 3 rounds of biopanning (FIG. 6C), and motif clustering analysis (FIG. 6D). The normalized frequency of each clone is represented in heatmaps. (FIG. 6E) Phage clone specificity validation by homology groups revealed preferential bindings of Groups 2 and 3 targeting clones to CP pancreas over the healthy pancreas. n=5. Mean±SEM. A student t-test was used to compare the targeting phage (VT680)-to-wild type phage (VT750) ratio in CP versus the same ratio in the healthy pancreas. *p<0.05 (p=0.0275 for Group 2; p=0.0443 for Group 3). (FIG. 6F) Phage clones from Groups 2 and 3 were validated individually in both healthy and CP mice. Fold change represents the ratio of targeting-to-wild type ratio in CP over healthy mice. 7/9 clones showed higher phage accumulation in CP over healthy pancreas (fold change>1).

FIG. 7. CP-homing phage clones show selectivity for cellular components in the CP pancreas. Immunofluorescence images of VT680-labeled phage colocalized with cell markers in the inflamed pancreas. Five cell markers were stained to represent five common cellular components in CP: Collagen IIIa (ECM; MDLSLKP; SEQ ID NO: 19), αSMA (activated PSC; KTYVPTT; SEQ ID NO: 14), F4/80 (macrophages; SNSQDLH; SEQ ID NO: 28), CPA-1 (acinar cells; MNSIAIP; SEQ ID NO: 20), CD31 (endothelium), and CK7 (epithelium). The quantitative evaluation of colocalization for all peptides against all cell markers is reported in Table 8. Colors in the merged images represent phage (green when shown in color) and cell markers (red when shown in color). Scale bar: 20 μm.

FIGS. 8A-8C. Pharmacokinetics of peptide-modified liposomes. (FIG. 8A) In vivo IVIS images of CP mice over 72 hour time course post-injection of peptide-modified liposomes. The white arrow indicates fluorescent signal detected at the pancreas region. (FIG. 8B) Ex vivo IVIS images of pancreas at 4, 48, and 72 hours post liposome injection. (FIG. 8C) Biodistribution of DiD-labeled liposomes in CP mice at 48 hours post injection. Fluorescent intensity is normalized to the number of particles injected, the number of DiD per liposome, and mass of the pancreas. n=3. Student t-test was used to compare the targeted liposomes to the no peptide liposomes. *p<0.05. SK M· Skeletal muscle.

FIGS. 9A and 9B. Immunofluorescence of peptide conjugated liposomes in CP pancreas. (FIG. 9A) NC liposomes were non-specifically taken up by macrophages presented in the inflamed pancreas. MDLSLKP (SEQ ID NO: 19) liposomes colocalized with extracellular matrix (collagen IIIa⁺ cells). Color code (used when shown in color): green for liposome (DiD), and red for cell markers. Scale bar: 20 μm. (FIG. 9B) Box-and whisker plot of MCC values of liposomes overlapping cell markers. Liposome selectivity for the corresponding cell types was analyzed using the ImageJ plug-in JACoP. n=7 to about 12 images per group. One-way ANOVA and Tukey-Kramer tests were used to compare MCC of all cell markers for each liposome. *p<0.05. No statistically significant difference was observed in spatial localization of MNSIAIP (SEQ ID NO: 20) liposomes with any stained cell types.

FIGS. 10A-10C. Apigenin loading in peptide-modified liposomes. (FIG. 10A) Chemical structure of apigenin. (FIG. 10B) Release profile of apigenin in PBS at 4C, pH 7.4, from liposomes. n=3. (FIG. 10C) In vitro release study in 50% FBS at 37° C. n=3.

FIGS. 11A-11G. Targeted delivery of Apigenin reduced fibrosis. (FIG. 11A) Schematic of CP mouse model followed by 3-week treatments of either empty ECM liposome (vehicle), free apigenin (free drug), apigenin-encapsulated naked liposomes (Api-Naked Lip), or apigenin-encapsulated MDLSLKP (SEQ ID NO: 19) liposomes (Api-ECM Lip; n=5). (FIG. 11B) H&E staining of pancreas by the end of 3-week treatments. A reduced interstitial space and acinar atrophy (indicated by arrows) was observed in the pancreas treated by targeted liposomes compared to free drug and the control liposomes. Scale bar: 50 μm. (FIG. 11C) Pancreas immunostained for fibronectin demonstrated targeted delivery of apigenin significantly decreased fibronectin expression. Scale bar: 100 μm. (FIG. 11D) Picrosirius red staining of pancreas Scale bar: 50 μm. (FIG. 11E) Number of acini atrophy found in a 256 μm×256 μm image. n=5 images/animal, 3 animals/group. (FIG. 11F) Quantification of fibronectin-positive area. n=8 images/animal, 5 animals/group. (FIG. 11G) Quantification of Picrosirius red-positive area. n=12 images/animal, 5 animals/group. In all images, ANOVA and Tukey test were used to compare Api-ECM Lip to the rest of the treatment groups. *p<0.05, ****p<0.0001.

FIGS. 12A-12C. Targeted liposomal delivery of apigenin reduces hepatotoxicity. (FIG. 12A) Chemistry test results generated from serum samples collected from vehicle, free drug-, Api-Naked Lip- and Api-ECM Lip-treated CP mice revealing statically significant reduction of toxic effects of the apigenin-based treatment to the mice livers (p=0.0196). No significant toxic effects induced to mice kidneys is observed in all treatments. Mean+/−SEM, n=3. (FIG. 12B) Representative H&E staining of livers. Scale bar: 100 μm. (FIG. 12C) Western blot images and the quantification of liver lysates from treated CP mice probing for SOD1. Mean+/−SEM, n=5, *p=0.0491.

### DETAILED DESCRIPTION

### I. General Considerations

Chronic pancreatitis (CP) is an inflammatory disorder that causes irreversible damage in the pancreas and induces long-standing sequelae encompassing recurrent severe pain, fibrosis, duct distortion, parenchymal calcification, and loss of exocrine and endocrine functions at the advanced stage (Klöppel, 1990; Etemad & Whitcomb, 2001). In addition to physical debilities, patients with CP typically struggle with psychological and financial challenges, resulting in significantly impaired quality of life measures (Morgan et al., 2012; Machicado et al., 2017). Patients with a history of CP have an increased risk of developing pulmonary diseases, diabetes mellitus, and pancreatic cancer (Bang et al., 2014). The annual incidence of CP worldwide ranges between S-12 per 100,000 population, with an approximate prevalence of 50 per 100,000 (Yadav & Lowenfels, 2013). Despite low prevalence, the frequent demands of pain management and necessary procedures directly or indirectly caused by CP lead to a disproportional high cost of medical care, inducing an impactful socioeconomic burden on diseased individuals and the health care system in the United States (Hall et al., 2014; Ting et al., 2016).

Fibrosis is a characteristic feature of CP, which is not only an outcome of recurrent pancreatic parenchymal cell necrosis but is also responsible for the post-injury reactions that induce a cascade of events and signifies molecular and cellular damage. Various cellular components and molecular crosstalk are involved in fibrogenesis, and each of the components contributes to the clinical outcome of pancreatic remodeling. However, no FDA-approved drug is available to address the fundamental causes of inflammation and fibrogenesis to halt and reverse the damages of pancreatitis. In fact, current therapeutic strategies for CP are limited to palliative care and pain alleviation, and these approaches fail at the advanced stage when invasive surgical procedures are the only available options. Thus, addressing fibrotic conditions has become one of the main focuses to improve pancreatitis outcomes. Apigenin (4′,5,7-trihydroxyflavone), a small molecule natural compound, has been shown preclinically to have antifibrotic, anti-inflammatory, antioxidant, and proapoptotic properties in CP and various cancers in vitro and in vivo (Shukla & Gupta, 2010; Mrazek et al., 2015; Mrazek et al., 2019). Despite these promising features, apigenin suffers from low aqueous solubility, metabolic instability, and off-target effects (Chen et al., 2014); and thus, no approved clinical applications of apigenin are available.

Liposome-based drug delivery has been clinically proven in the cancer setting to successfully encapsulate small molecule drugs for enhanced bioavailability, prolonged drug circulation half-life, and improved patient outcomes (Bornmann et al., 2008). Surface modifications with targeting ligands such as antibodies, antibody fragments, and peptides enable specific binding at the diseased sites while minimizing undesired side-effects induced by off-targeting. In the tissue remodeling process of CP, various cellular and molecular components are involved and contributed to the progression, which make them potential targets. There are, however, no molecular targeting ligands or non-serum-based biomarkers available, limiting the development of imaging agents and therapeutics for pancreatitis.

To fill that void, we used phage display and our innovative and computational guided target selection approach, PHASTpep (Brinton et al., 2016), to identify peptides specific for key cellular components involved in fibrogenesis. Through in vivo imaging and colocalization analysis, we have demonstrated cell specificity of five 7-mer peptides for acinar cells, activated pancreatic stellate cells (aPSCs), extracellular matrix (ECM), and macrophages present in the CP microenvironment In a representative embodiment, we developed an apigenin-encapsulated liposomal formulation with surface modified with the ECM specific peptide MDLSLKP (SEQ ID NO: 19), and demonstrated ligand-mediated drug delivery to the inflamed pancreas. After three weeks of treatment, we demonstrated a 37.2% and 33.1% reduction in collagen deposition and fibronectin expression, respectively, in the liposomal preparation compared to the free drug form. The enhanced remodeling outcomes of the MDLSKLP (SEQ ID NO: 19) conjugated compared to the no-ligand liposomes and free drug indicates that for chronic pancreatitis cell type-specific targeting improved antifibrotic efficacy of a small molecule drug, which had undesirable properties. Disclosed herein is the use of a high-throughput ligand selection approach to identify peptides specific for CP and proved the application of these targeting peptides in drug delivery. The presently disclosed subject matter thus provides tools to mediate the development of target-specific interventions for CP.

In accordance with aspects of the presently disclosed subject matter, a computational-guided in vivo phage display approach was utilized to select peptide ligands specific for cellular components in the caerulein-induced mouse model of CP. Peptides specific for activated pancreatic stellate cells, acinar cells, macrophages, and extracellular matrix were identified. The collagen IIIa⁺ cell targeted peptide was conjugated to liposomes and targeted delivery of an antifibrotic small molecule drug, apigenin, was demonstrated. After 3 weeks of treatment, acini preservation and stromal-fibrosis-reduction were observed. There was a 37.2% and 33.1% respective reduction in collagen and fibronectin expression in mice receiving apigenin-encapsulated targeted liposomes compared to the free drug. The presently disclosed subject matter will now be described more fully. The presently disclosed subject matter can, however, be embodied in different forms and should not be construed as limited to the embodiments set forth herein below and in the accompanying Examples. Rather, these embodiments are provided so that this disclosure will be thorough and complete, and will fully convey the scope of the embodiments to those skilled in the art

### II. Definitions

While the following terms are believed to be well understood by one of ordinary skill in the art, the following definitions are set forth to facilitate explanation of the presently disclosed subject matter.

Unless defined otherwise, all technical and scientific terms used herein have the same meaning as commonly understood to one of ordinary skill in the art to which the presently disclosed subject matter belongs.

Following long-standing patent law convention, the terms “a”, “an”, and “the” refer to “one or more” when used in this application, including the claims.

The term “and/or” when used in describing two or more items or conditions, refers to situations where all named items or conditions are present or applicable, or to situations wherein only one (or less than all) of the items or conditions is present or applicable.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” can mean at least a second or more.

The term “comprising”, which is synonymous with “including,” “containing,” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

Unless otherwise indicated, all numbers expressing quantities of time, concentration, dosage and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about”, when referring to a value is meant to encompass variations of in one example ±20% or ±10%, in another example ±5%, in another example ±1%, and in still another example ±0.1% from the specified amount, as such variations are appropriate to perform the disclosed methods.

As use herein, the terms “administration of” and/or “administering” a compound or composition can be understood to refer to providing a compound or composition (e.g., targeted liposomes comprising an active agent, such as a drug) of the presently disclosed subject matter to a subject in need of treatment. As used herein “administering” includes administration of a compound or composition by any number of routes and modes including, but not limited to, topical, oral, buccal, intravenous, intramuscular, intra-arterial, intramedullary, intrathecal, intraventricular, transdermal, subcutaneous, intraperitoneal, intranasal, enteral, topical, sublingual, vaginal, ophthalmic, pulmonary, vaginal, and rectal approaches.

As used herein, an “effective amount” or “therapeutically effective amount” refers to an amount of a compound or composition sufficient to produce a selected effect, such as but not limited to alleviating symptoms of a condition, disease, or disorder. In the context of administering a compound or composition in the form of a combination, such as multiple compositions, the amount of each a compound or composition, when administered in combination with one or more other compositions, may be different from when that composition is administered alone. Thus, an effective amount of a combination of compounds or compositions refers collectively to the combination as a whole, although the actual amounts of each a compound or composition may vary. The term “more effective” means that the selected effect occurs to a greater extent by one treatment relative to the second treatment to which it is being compared.

The term “prevent”, as used herein, means to stop something from happening, or taking advance measures against something possible or probable from happening. In the context of medicine, “prevention” generally refers to action taken to decrease the chance of getting a disease or condition. It is noted that “prevention” need not be absolute, and thus can occur as a matter of degree.

The terms “treatment” and “treating” as used herein refer to both therapeutic treatment and prophylactic or preventative measures, wherein the object is to prevent or slow down (lessen) the targeted pathologic condition, prevent the pathologic condition, pursue or obtain beneficial results, and/or lower the chances of the individual developing a condition, disease, or disorder, even if the treatment is ultimately unsuccessful. Those in need of treatment include those already with the condition as well as those prone to have or predisposed to having a condition, disease, or disorder, or those in whom the condition is to be prevented.

The methods and compositions disclosed herein can be used on a sample either in vitro (for example, on isolated cells or tissues) or in vivo in a subject (i.e. living organism, such as a patient). In some embodiments, the subject is a human subject, although it is to be understood that the principles of the presently disclosed subject matter indicate that the presently disclosed subject matter is effective with respect to all vertebrate species, including mammals, which are intended to be included in the terms “subject” and “patient”. Moreover, a mammal is understood to include any mammalian species for which employing the compositions and methods disclosed herein is desirable, particularly agricultural and domestic mammalian species.

As such, the methods of the presently disclosed subject matter are particularly useful in warm-blooded vertebrates. Thus, the presently disclosed subject matter concerns mammals and birds. More particularly provided are methods and compositions for mammals such as humans, as well as those mammals of importance due to being endangered (such as Siberian tigers), of economic importance (animals raised on farms for consumption by humans), and/or of social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Also provided is the treatment of birds, including the treatment of those kinds of birds that are endangered, kept in zoos or as pets (e.g., parrots), as well as fowl, and more particularly domesticated fowl, for example, poultry, such as turkeys, chickens, ducks, geese, guinea fowl, and the like, as they are also of economic importance to humans. Thus, also provided is the treatment of livestock including, but not limited to domesticated swine (pigs and hogs), ruminants, horses, poultry, and the like.

As used herein, an “analog” of a chemical compound is a compound that, by way of example, resembles another in structure but is not necessarily an isomer (e.g., 5-fluorouracil is an analog of thymine).

As used herein, amino acids are represented by the full name thereof, by the three letter code corresponding thereto, and/or by the one-letter code corresponding thereto, as summarized in Table 1:

TABLE 1
Amino Acids and Codes Therefor

| | 3-Letter | 1-Letter | | 3-Letter | 1-Letter |
|---|---|---|---|---|---|
| Full Name | Code | Code | Full Name | Code | Code |
| Aspartic Acid | Asp | D | Threonine | Thr | T |
| Glutamic Acid | Glu | E | Glycine | Gly | G |
| Lysine | Lys | K | Alanine | Ala | A |
| Arginine | Arg | R | Valine | Val | V |
| Histidine | His | H | Leucine | Leu | L |
| Tyrosine | Tyr | Y | Isoleucine | Ile | I |
| Cysteine | Cys | C | Methionine | Met | M |
| Asparagine | Asn | N | Proline | Pro | P |
| Glutamine | Gln | Q | Phenylalanine | Phe | F |
| Serine | Ser | S | Tryptophan | Trp | W |

The expression “amino acid” as used herein is meant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides of the presently disclosed subject matter, and particularly at the carboxy- or amino-terminus, can be modified by methylation, amidation, acetylation or substitution with other chemical groups which can change the peptide's circulating half-life without adversely affecting their activity. Additionally, a disulfide linkage may be present or absent in the peptides of the presently disclosed subject matter. The term “amino acid” is used interchangeably with “amino acid residue”, and may refer to a free amino acid and to an amino acid residue of a peptide. It will be apparent from the context in which the term is used whether it refers to a free amino acid or a residue of a peptide.

Amino acids have the following general structure:

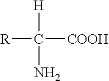

Amino acids may be classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

The nomenclature used to describe the peptide compounds of the presently disclosed subject matter follows the conventional practice wherein the amino group is presented to the left and the carboxy group to the right of each amino acid residue. In the formulae representing selected specific embodiments of the presently disclosed subject matter, the amino-and carboxy-terminal groups, although not specifically shown, will be understood to be in the form they would assume at physiologic pH values, unless otherwise specified.

As used herein, the term “conservative amino acid substitution” is defined herein as an amino acid exchange within one of the five groups summarized in Table 2. It is noted that in some embodiments, a “derivative” of a reference amino acid sequence is an amino acid sequence that, as compared to the reference amino acid sequence, includes one or more conservative amino acid substitutions.

TABLE 2
Conservative Amino Acid Substitutions

| Group | Characteristics | Amino Acids |
|---|---|---|
| A. | Small aliphatic, nonpolar | Ala, Ser, Thr, Pro, Gly |
| | or slightly polar residues | |
| B. | Polar, negatively charged | Asp, Asn, Glu, Gln |
| | residues and their amides | |
| C. | Polar, positively charged | His, Arg, Lys |
| | residues | |
| D. | Large, aliphatic, nonpolar | Met Leu, Ile, Val, Cys |
| | residues | |
| E. | Large, aromatic residues | Phe, Tyr, Trp |

As used herein, the term “mimetic” refers to a compound having similar functional and/or structural properties to another known compound or a particular fragment of that known compound, such as a known compound of biological origin, e.g., a peptide. As such, the phrase “peptide mimetic” as used herein refers to a compound at least a part of which includes a non-peptide structure that has similar functional and/or structural properties to a peptide of the presently disclosed subject matter. In some embodiments, the similar functional and/or structural properties is a binding activity to a target to which a peptide of the presently disclosed subject matter binds.

The term “nanoparticle” refers to a structure having at least one region with a dimension (e.g., length, width, diameter, etc.) of less than about 1,000 nm. In some embodiments, the dimension is smaller (e.g., less than about 500 nm, less than about 250 nm, less than about 200 nm, less than about 150 nm, less than about 125 nm, less than about 100 nm, less than about 80 nm, less than about 70 nm, less than about 60 nm, less than about 50 nm, less than about 40 nm, less than about 30 nm or even less than about 20 nm). In some embodiments, the dimension is between about 20 nm and about 250 nm (e.g., about 20, 30, 40, 50, 60, 70, 80, 90, 100, 110, 120, 130, 140, 150, 160, 170, 180, 190, 200, 210, 220, 230, 240, or 250 nm). In some embodiments, a nanoparticle is approximately spherical. When the nanoparticle is approximately spherical, the characteristic dimension can correspond to the diameter of the sphere. In addition to spherical shapes, the nanomaterial can be disc-shaped, plate-shaped (e.g., hexagonally plate-like), oblong, polyhedral, rod-shaped, cubic, or irregularly-shaped. The nanoparticle can comprise a core region (i.e., the space between the outer dimensions of the particle) and an outer surface (i.e., the surface that defines the outer dimensions of the particle). In some embodiments, the nanoparticle can have one or more coating layers surrounding or partially surrounding the nanoparticle core. Thus, for example, a spherical nanoparticle can have one or more concentric coating layers, each successive layer being dispersed over the outer surface of a smaller layer closer to the center of the particle. Such nanoparticles can be referred to as “core-shell” nanoparticles, wherein the shell refers to the coating layer or layers. In some embodiments, an active agent is encapsulated by the nanoparticle, and in some embodiments an active agent is otherwise associated with the nanoparticle, including but not limited to being attached to a surface (e.g., an outer surface) of the nanoparticle, which can include covalent and/or non-covalent attachments. In some embodiments, the nanoparticle is designed to release the active agent once the nanoparticle localizes at a target of interest.

The term “peptide” typically refers to short polypeptides.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” means a non-naturally occurring peptide or polypeptide, and can include recombinant polypeptides Synthetic peptides or polypeptides can be synthesized, for example, using an automated polypeptide synthesizer. Various solid phase peptide synthesis methods are known to those of skill in the art.

The term “protein” typically refers to large polypeptides. Conventional notation is used herein to portray polypeptide sequences: the left-hand end of a polypeptide sequence is the amino-terminus; the right-hand end of a polypeptide sequence is the carboxyl-terminus.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure. Representative purification techniques are disclosed herein for antibodies and fragments thereof.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide may serve a non-coding function (e.g., promoter, origin of replication, ribosome-binding site, etc.) as well.

A host cell that comprises a recombinant polynucleotide is referred to as a “recombinant host cell”. A gene which is expressed in a recombinant host cell wherein the gene comprises a recombinant polynucleotide, produces a “recombinant polypeptide”.

A “recombinant polypeptide” is one which is produced upon expression of a recombinant polynucleotide.

### Peptide Modification and Preparation

Methods and reagents for peptide production are known in the art. It will be appreciated, of course, that the proteins or peptides of the presently disclosed subject matter may incorporate amino acid residues which are modified without affecting activity. For example, the termini may be derivatized to include blocking groups, i.e., chemical substituents suitable to protect and/or stabilize the N- and C-termini from “undesirable degradation”, a term meant to encompass any type of enzymatic, chemical or biochemical breakdown of the compound at its termini which is likely to affect the function of the compound, i.e. sequential degradation of the compound at a terminal end thereof.

Blocking groups include protecting groups conventionally used in the art of peptide chemistry which will not adversely affect the in vivo activities of the peptide. For example, suitable N-terminal blocking groups can be introduced by alkylation or acylation of the N-terminus. Examples of suitable N-terminal blocking groups include C₁-C₅ branched or unbranched alkyl groups, acyl groups such as formyl and acetyl groups, as well as substituted forms thereof, such as the acetamidomethyl (Acm) group. Desamino analogs of amino acids are also useful N-terminal blocking groups, and can either be coupled to the N-terminus of the peptide or used in place of the N-terminal reside. Suitable C-terminal blocking groups, in which the carboxyl group of the C-terminus is either incorporated or not, include esters, ketones or amides. Ester or ketone-forming alkyl groups, particularly lower alkyl groups such as methyl, ethyl and propyl, and amide-forming amino groups such as primary amines (—NH₂), and mono- and di-alkylamino groups such as methylamino, ethylamino, dimethylamino, diethylamino, methylethylamino and the like are examples of C-terminal blocking groups. Descarboxylated amino acid analogues such as agmatine are also useful C-terminal blocking groups and can be either coupled to the peptide's C-terminal residue or used in place of it. Further, it will be appreciated that the free amino and carboxyl groups at the termini can be removed altogether from the peptide to yield desamino and descarboxylated forms thereof without affect on peptide activity.

Acid addition salts of the presently disclosed subject matter are also contemplated as functional equivalents. Thus, a peptide in accordance with the presently disclosed subject matter treated with an inorganic acid such as hydrochloric, hydrobromic, sulfuric, nitric, phosphoric, and the like, or an organic acid such as an acetic, propionic, glycolic, pyruvic, oxalic, malic, malonic, succinic, maleic, fumaric, tataric, citric, benzoic, cinnamie, mandelic, methanesulfonic, ethanesulfonic, p-toluenesulfonic, salicyclic and the like, to provide a water soluble salt of the peptide is suitable for use in the presently disclosed subject matter.

The presently disclosed subject matter also provides for analogs of proteins. Analogs can differ from naturally occurring proteins or peptides by conservative amino acid sequence differences or by modifications which do not affect sequence, or by both. For example, conservative amino acid changes may be made, which although they alter the primary sequence of the protein or peptide, do not normally alter its function. To that end, one, two, or sometimes more, conservative amino acid changes typically have no effect on peptide function.

Modifications (which do not normally alter primary sequence) include in vivo, or in vitro chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

Also included are polypeptides which have been modified using ordinary molecular biological techniques so as to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring or non-standard synthetic amino acids. The peptides of the presently disclosed subject matter are not limited to products of any of the specific exemplary processes listed herein.

The presently disclosed subject matter includes the use of beta-alanine (also referred to as β-alanine, β-Ala, bA, and βA, having the structure:

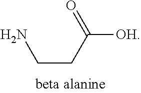

It will be appreciated, of course, that the peptides or antibodies, derivatives, or fragments thereof may incorporate amino acid residues which are modified without affecting activity. For example, the termini may be derivatized to include blocking groups, i.e. chemical substituents suitable to protect and/or stabilize the N- and C-termini from “undesirable degradation”, a term meant to encompass any type of enzymatic, chemical or biochemical breakdown of the compound at its termini which is likely to affect the function of the compound, i.e. sequential degradation of the compound at a terminal end thereof.

Other modifications can also be incorporated without adversely affecting the activity and these include, but are not limited to, substitution of one or more of the amino acids in the natural L-isomeric form with amino acids in the D-isomeric form. Thus, the peptide may include one or more D-amino acid resides, or may comprise amino acids which are all in the D-form. Retro-inverso forms of peptides in accordance with the presently disclosed subject matter are also contemplated, for example, inverted peptides in which all amino acids are substituted with D-amino acid forms.

Substantially pure protein obtained as described herein may be purified by following known procedures for protein purification, wherein an immunological, enzymatic or other assay is used to monitor purification at each stage in the procedure. Protein purification methods are well known in the art, and are described, for example in Deutscher et al., 1990.

As discussed, modifications or optimizations of peptide ligands of the presently disclosed subject matter are within the scope of the application. Modified or optimized peptides are included within the definition of peptide binding ligand. Specifically, a peptide sequence identified can be modified to optimize its potency, pharmacokinetic behavior, stability and/or other biological, physical and chemical properties.

### Amino Acid Substitutions

In certain embodiments, the disclosed methods and compositions may involve preparing peptides with one or more substituted amino acid residues.

In various embodiments, the structural, physical and/or therapeutic characteristics of peptide sequences may be optimized by replacing one or more amino acid residues.

Other modifications can also be incorporated without adversely affecting the activity and these include, but are not limited to, substitution of one or more of the amino acids in the natural L-isomeric form with amino acids in the D-isomeric form. Thus, the peptide may include one or more D-amino acid resides, or may comprise amino acids which are all in the D-form. Retro-inverso forms of peptides in accordance with the presently disclosed subject matter are also contemplated, for example, inverted peptides in which all amino acids are substituted with D-amino acid forms.

The skilled artisan will be aware that, in general, amino acid substitutions in a peptide typically involve the replacement of an amino acid with another amino acid of relatively similar properties (i.e., conservative amino acid substitutions). The properties of the various amino acids and effect of amino acid substitution on protein structure and function have been the subject of extensive study and knowledge in the art.

For example, one can make the following isosteric and/or conservative amino acid changes in the parent polypeptide sequence with the expectation that the resulting polypeptides would have a similar or improved profile of the properties described above:

Substitution of alkyl-substituted hydrophobic amino acids. including alanine, leucine, isoleucine, valine, norleucine, S-2-aminobutyric acid, S-cyclohexylalanine or other simple alpha-amino acids substituted by an aliphatic side chain from C₁₋₁₀ carbons including branched, cyclic and straight chain alkyl, alkenyl or alkynyl substitutions.

Substitution of aromatic-substituted hydrophobic amino acids: including phenylalanine, tryptophan, tyrosine, biphenylalanine, 1-naphthylalanine, 2-naphthylalanine, 2-benzothienylalanine, 3-benzothienylalanine, histidine, amino, alkylamino, dialkylamino, aza, halogenated (fluoro, chloro, bromo, or iodo) or alkoxy-substituted forms of the previous listed aromatic amino acids, illustrative examples of which are: 2-,3- or 4-aminophenylalanine, 2-,3- or 4-chlorophenylalanine, 2-,3- or 4-methylphenylalanine, 2-,3- or 4-methoxyphenylalanine, 5-amino-, 5-chloro-, 5-methyl- or 5-methoxytryptophan, 2′-, 3′-, or 4′-amino-, 2′-, 3′-, or 4′-chloro-, 2,3, or 4-biphenylalanine, 2′-,3′-, or 4′-methyl-2, 3 or 4-biphenylalanine, and 2- or 3-pyridylalanine.

Substitution of amino acids containing basic functions: including arginine, lysine, histidine, ornithine, 2,3-diaminopropionic acid, homoarginine, alkyl, alkenyl, or aryl-substituted (from C₁-C₁₀ branched, linear, or cyclic) derivatives of the previous amino acids, whether the substituent is on the heteroatoms (such as the alpha nitrogen, or the distal nitrogen or nitrogens, or on the alpha carbon, in the pro-R position for example. Compounds that serve as illustrative examples include: N-epsilon-isopropyl-lysine, 3-(4-tetrahydropyridyl)-glycine, 3-(4-tetrahydropyridyl)-alanine, N,N-gamma, gamma'-diethyl-homoarginine. Included also are compounds such as alpha methyl arginine, alpha methyl 2,3-diaminopropionic acid, alpha methyl histidine, alpha methyl ornithine where alkyl group occupies the pro-R position of the alpha carbon. Also included are the amides formed from alkyl, aromatic, heteroaromatic (where the heteroaromatic group has one or more nitrogens, oxygens, or sulfur atoms singly or in combination) carboxylic acids or any of the many well-known activated derivatives such as acid chlorides, active esters, active azolides and related derivatives) and lysine, ornithine, or 2,3-diaminopropionic acid.

Substitution of acidic amino acids: including aspartic acid, glutamic acid, homoglutamic acid, tyrosine, alkyl, aryl, arylalkyl, and heteroaryl sulfonamides of 2,4-diaminopriopionic acid, ornithine or lysine and tetrazole-substituted alkyl amino acids.

Substitution of side chain amide residues: including asparagine, glutamine, and alkyl or aromatic substituted derivatives of asparagine or glutamine.

Substitution of hydroxyl containing amino acids: including serine, threonine, homoserine, 2,3-diaminopropionic acid, and alkyl or aromatic substituted derivatives of serine or threonine. It is also understood that the amino acids within each of the categories listed above can be substituted for another of the same group.

For example, the hydropathic index of amino acids may be considered (Kyte & Doolittle, 1982). The relative hydropathic character of the amino acid contributes to the secondary structure of the resultant protein, which in turn defines the interaction of the protein with other molecules. Each amino acid has been assigned a hydropathic index on the basis of its hydrophobicity and charge characteristics (Kyte & Doolittle, 1982), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). In making conservative substitutions, amino acids whose hydropathic indices are within ±2 can in some embodiments be used, within ±1 can in some embodiments be used, and in some embodiments amino acids with hydropathic indices within ±0.5 can be used.

Amino acid substitution may also take into account the hydrophilicity of the amino acid residue (e.g., U.S. Pat. No. 4,554,101). Hydrophilicity values have been assigned to amino acid residues: arginine (+3.0); lysine (+3.0); aspartate (+3.0); glutamate (+3.0); serine (+0.3); asparagine (+0.2); glutamine (+0.2); glycine (0); threonine (−0.4); proline (−0.5±0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). In some embodiments, replacement of amino acids with others of similar hydrophilicity is employed.

Other considerations include the size of the amino acid side chain. For example, in some embodiments replacement of an amino acid with a compact side chain, such as glycine or serine, with an amino acid with a bulky side chain, e.g., tryptophan or tyrosine is less desirable. The effect of various amino acid residues on protein secondary structure is also a consideration. Through empirical study, the effect of different amino acid residues on the tendency of protein domains to adopt an alpha-helical, beta-sheet or reverse turn secondary structure has been determined and is known in the art (see e.g., Chou & Fasman, 1974; Chou & Fasman, 1978; Chou & Fasman, 1979).

Based on such considerations and extensive empirical study, tables of conservative amino acid substitutions have been constructed and are known in the art. For example: arginine and lysine; glutamate and aspartate; serine and threonine; glutamine and asparagine; and valine, leucine and isoleucine. Alternatively: Ala (A) Leu, Ile, Val; Arg (R) Gln, Asn, Lys; Asn (N) His, Asp, Lys, Arg, Gln; Asp (D) Asn, Glu; Cys (C) Ala, Ser; Gln (Q) Glu, Asn; Glu (E) Gln, Asp; Gly (G) Ala; His (H) Asn, Gln, Lys, Arg; Ile (I) Val, Met, Ala, Phe, Leu; Leu (L) Val, Met, Ala, Phe, Ile; Lys (K) Gln, Asn, Arg; Met (M) Phe, Ile, Leu; Phe (F) Leu, Val, Ile, Ala, Tyr, Pro (P) Ala; Ser (S), Thr; Thr (T) Ser; Trp (W) Phe, Tyr; Tyr (Y) Trp, Phe, Thr, Ser; Val (V) Ile, Leu, Met, Phe, Ala.

Other considerations for amino acid substitutions include whether or not the residue is located in the interior of a protein or is solvent exposed. For interior residues, conservative substitutions would include: Asp and Asn; Ser and Thr; Ser and Ala; Thr and Ala; Ala and Gly; Ile and Val; Val and Leu; Leu and Ile; Leu and Met; Phe and Tyr; Tyr and Trp. See e.g., PROWL Rockefeller University website. For solvent exposed residues, conservative substitutions would include: Asp and Asn; Asp and Glu, Glu and Gln; Glu and Ala, Gly and Asn; Ala and Pro; Ala and Gly; Ala and Ser; Ala and Lys; Ser and Thr; Lys and Arg; Val and Leu; Leu and Ile; Ile and Val; Phe and Tyr. Various matrices have been constructed to assist in selection of amino acid substitutions, such as the PAM250 scoring matrix, Dayhoff matrix, Grantham matrix, Mclachlan matrix, Doolittle matrix, Henikoff matrix, Miyata matrix, Fitch matrix, Jones matrix, Rao matrix, Levin matrix and Risler matrix.

In determining amino acid substitutions, one may also consider the existence of intermolecular or intramolecular bonds, such as formation of ionic bonds (salt bridges) between positively charged residues (e.g., His, Arg, Lys) and negatively charged residues (e.g., Asp, Glu) or disulfide bonds between nearby cysteine residues.

Methods of substituting any amino acid for any other amino acid in an encoded peptide sequence are well known and a matter of routine experimentation for the skilled artisan, for example by the technique of site-directed mutagenesis or by synthesis and assembly of oligonucleotides encoding an amino acid substitution and splicing into an expression vector construct.

### IV. Compositions

### IV.A. Generally

In some embodiments, the presently disclosed subject matter relates to peptides that have been identified as binding to various targets, including but not limited to biological targets

In some embodiments, a peptide of the presently disclosed subject matter comprises, consists essentially of, or consists of an amino acid sequence as set forth in any one of SEQ ID NOs: 3-38 or a mimetic, analog, or derivative thereof, optionally wherein the amino acid sequence is no more than 10 amino acids, 20 amino acids, 30 amino acids, 40 amino acids, or 50 amino acids. In some embodiments, one or more of the amino acids in the amino acid sequence of a peptide of the presently disclosed subject matter is a modified amino acid and/or a non-standard amino acid. It is understood that with respect to the amino peptides disclosed herein, the modified amino acid(s) and/or non-standard amino acid(s) can be one of the amino acids set forth in any one of SEQ ID NOs: 3-38. Similarly, when a peptide comprises or consists essentially of one of SEQ ID NOs: 3-38, the amino acid sequences set forth in SEQ ID NOs: 3-38 can be unmodified (i.e., the modified amino acid(s) and/or non-standard amino acid(s) in a position N-terminal and/or C-terminal to the amino acid sequences set forth in SEQ ID NOs: 3-38.

In some embodiments, a peptide of the presently disclosed subject matter is conjugated or otherwise associated with a delivery vehicle to thereby target and/or deliver the delivery vehicle to a target of interest. Particular amino acids that have targeting activity include, but are not limited to the peptides KTYVPTT (SEQ ID NO: 14), MDLSLKP (SEQ ID NO: 19), MNSIAIP (SEQ ID NO: 20) , SLINSSF (SEQ ID NO: 27), and SNSQDLH (SEQ ID NO: 28).

In some embodiments, a peptide of the presently disclosed subject matter is conjugated to and/or associated with a delivery vehicle. Examples of delivery vehicles are liposomes, mixed liposomes, oleosomes, niosomes, ethosomes, milliparticles, microparticles, nanoparticles and solid lipid nanoparticles, nanostructured lipid carriers, sponges, cyclodextrins, vesicles, micelles, mixed micelles of surfactants, mixed micelles of surfactant-phospholipid, millispheres, microspheres and nanospheres, lipospheres, millicapsules, microcapsules and nanocapsules, as well as microemulsions and nanoemulsions, which can be added to achieve a greater penetration of the active principle and/or improve its pharmacokinetic and pharmacodynamic properties. Exemplary delivery vehicles are liposomes, which can be conjugated to the peptides of the presently disclosed subject matter using standard techniques and as described herein.

Thus, in some embodiments the presently disclosed subject matter relates to liposomal compositions comprising a liposome, wherein a surface of the liposome has been conjugated to or is other wise associated with a peptide of the presently disclosed subject matter, including but not limited to peptides comprising, consisting essentially of, or consisting of an amino acid sequence as set forth in any of SEQ ID NOs: 3-38.

As described in more detail herein below, the peptides of the presently disclosed subject matter can be used to target the delivery vehicles of the presently disclosed subject matter to specific biological targets. By way of example and not limitation, a peptide that comprises, consists essentially of, or consists of SEQ ID NO: 19 can bind to a collagen III antigen, optionally a collagen III antigen that is present in the extracellular matrix of a cell; a peptide that comprises, consists essentially of, or consists of SEQ ID NO: 20 can bind to a carboxypeptidase A1 (CPA-1) antigen, optionally a CPA-1 antigen that is present on a pancreatic acinar cell; a peptide that comprises, consists essentially of, or consists of SEQ ID NO: 14 can bind to an α-smooth muscle actin (α-SMA) antigen, optionally an α-SMA antigen that is present on a cell; and a peptide that comprises, consists essentially of, or consists of SEQ ID NO: 27 and/or SEQ ID NO: 28 can bind to an F4/80 antigen, optionally an F4/80 antigen that is present on a cell. Thus, delivery vehicles that incorporate these peptides can be used to deliver active agents, such as but not limited to active agents that are complexed to and/or encompassed by delivery vehicles such as liposomes, to targets that express one or more of a collagen III antigen, a CPA-1 antigen, and α-SMA antigen, and/or an F4/80 antigen. The targeting can be accomplished in vitro, ex vivo, and/or in vivo.

In some embodiments, the peptide is MDLSLKP (SEQ ID NO: 19) and the delivery vehicle (e.g., a liposome) is targeted to a cell expressing collagen IIIa. In some embodiments, an MDLSLKP (SEQ ID NO: 19) conjugated and/or associated delivery vehicle (e.g., liposome) can be used to target collagen IIIa in the extracellular matrix.

Any active agent can be employed with (e.g., encapsulated by) the delivery vehicles (e.g., liposomes) of the presently disclosed subject matter. Exemplary active agents include, but are not limited to detectable agents such as but not limited to fluorescent agents and radioactive agents (e.g., for cell/tissue/organ visualization methods) and therapeutic agents including but not limited to chemotherapeutic agents and antifibrotic agents. An exemplary antifibrotic agent that can be employed in the compositions of the presently disclosed subject matter is apigenin (4′,5,7-trihydroxyflavone).

### IV.B. Formulations

Compositions as described herein comprise in some embodiments a composition that includes a pharmaceutically acceptable carrier. Suitable formulations include aqueous and non-aqueous sterile injection solutions that can contain antioxidants, buffers, bacteriostats, bactericidal antibiotics, and solutes that render the formulation isotonic with the bodily fluids of the intended recipient; and aqueous and non-aqueous sterile suspensions, which can include suspending agents and thickening agents. In some embodiments, a formulation of the presently disclosed subject matter comprises an adjuvant, optionally an oil-based adjuvant.

The compositions used in the methods can take such forms as suspensions, solutions, or emulsions in oily or aqueous vehicles, and can contain formulatory agents such as suspending, stabilizing, and/or dispersing agents. The compositions used in the methods can take forms including, but not limited to perioral, intravenous, intraperitoneal, intramuscular, and intratumoral formulations. Alternatively or in addition, the active ingredient can be in powder form for constitution with a suitable vehicle (e.g., sterile pyrogen-free water) before use.

The formulations can be presented in unit-dose or multi-dose containers, for example sealed ampules and vials, and can be stored in a frozen or freeze-dried (lyophilized) condition requiring only the addition of sterile liquid carrier immediately prior to use.

For oral administration, the compositions can take the form of, for example, tablets or capsules prepared by a conventional technique with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycollate); or wetting agents (e.g., sodium lauryl sulfate). The tablets can be coated by methods known in the art. For example, a neuroactive steroid can be formulated in combination with hydrochlorothiazide, and as a pH stabilized core having an enteric or delayed-release coating which protects the neuroactive steroid until it reaches the colon.

Liquid preparations for oral administration can take the form of, for example, solutions, syrups or suspensions, or they can be presented as a dry product for constitution with water or other suitable vehicle before use. Such liquid preparations can be prepared by conventional techniques with pharmaceutically acceptable additives such as suspending agents (e.g., sorbitol syrup, cellulose derivatives or hydrogenated edible fats); emulsifying agents (e g. lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring, and sweetening agents as appropriate. Preparations for oral administration can be suitably formulated to give controlled release of the active compound. For buccal administration the compositions can take the form of tablets or lozenges formulated in conventional manner.

The compounds can also be formulated as a preparation for implantation or injection. Thus, for example, the compounds can be formulated with suitable polymeric or hydrophobic materials (e.g., as an emulsion in an acceptable oil) or ion exchange resins, or as sparingly soluble derivatives (e.g., as a sparingly soluble salt).

The compounds can also be formulated in oils that are administered as water-in-oil emulsions, oil-in-water emulsions, or water-in-oil-in water emulsions.

The compounds can also be formulated in rectal compositions (e.g., suppositories or retention enemas containing conventional suppository bases such as cocoa butter or other glycerides), creams or lotions, or transdermal patches.

In some embodiments, the presently disclosed subject matter employs a composition that is pharmaceutically acceptable for use in humans. One of ordinary skill in the art understands the nature of those components that can be present in such a composition that is pharmaceutically acceptable for use in humans and also what components should be excluded from compositions that are pharmaceutically acceptable for use in humans.

### V.C. Doses

As used herein, the phrases “treatment effective amount”, “therapeutically effective amount”, “treatment amount”, and “effective amount” are used interchangeably and refer to an amount of a therapeutic composition sufficient to produce a measurable response (e.g., a biologically or clinically relevant response in a subject being treated). Actual dosage levels of active ingredients in the pharmaceutical compositions of the presently disclosed subject matter can be varied so as to administer an amount of the active compound(s) that is effective to achieve the desired therapeutic response for a particular subject. The selected dosage level can depend upon the activity of the therapeutic composition, the route of administration, combination with other drugs or treatments, the severity of the condition being treated, the condition and prior medical history of the subject being treated, etc. However, it is within the skill of the art to start doses of the compound at levels lower than required to achieve the desired therapeutic effect and to gradually increase the dosage until the desired effect is achieved.

The potency of a therapeutic composition can vary, and therefore a “therapeutically effective amount” can vary. However, one skilled in the art can readily assess the potency and efficacy of a candidate modulator of the presently disclosed subject matter and adjust the therapeutic regimen accordingly.

After review of the disclosure herein of the presently disclosed subject matter, one of ordinary skill in the art can tailor the dosages to an individual subject, taking into account the particular formulation, method of administration to be used with the composition, and other factors. Further calculations of dose can consider subject height and weight, severity and stage of symptoms, and the presence of additional deleterious physical conditions. Such adjustments or variations, as well as evaluation of when and how to make such adjustments or variations, are well known to those of ordinary skill in the art of medicine.

For administration of a composition as disclosed herein, conventional methods of extrapolating human dosage based on doses administered to a murine animal model can be carried out using techniques known to one of ordinary skill in the art. Drug doses can also be given in milligrams per square meter of body surface area because this method rather than body weight achieves a good correlation to certain metabolic and excretionary functions. Moreover, body surface area can be used as a common denominator for drug dosage in adults and children as well as in different animal species as described by Freireich et al., 1966. Briefly, to express a mg/kg dose in any given species as the equivalent mg/m2 dose, multiply the dose by the appropriate km factor. In an adult human, 100 mg/kg is equivalent to 100 mg/kg×37 kg/m²=3700 mg/m².

For additional guidance regarding formulations and doses, see U.S. Pat. Nos. 5,326,902; 5,234,933; PCT International Publication No. WO 93/25521, Remington et al., 1975; Goodman et al., 1996; Berkow et al., 1997; Speight et al., 1997; Ebadi, 1998; Duch et al , 1998; Katzung, 2001; Gerbino, 2005.

### V.D. Routes of Administration

The presently disclosed compositions can be administered to a subject in any form and/or by any route of administration. In some embodiments, the formulation is a sustained release formulation, a controlled release formulation, or a formulation designed for both sustained and controlled release. As used herein, the term “sustained release” refers to release of an active agent such that an approximately constant amount of an active agent becomes available to the subject over time. The phrase “controlled release” is broader, referring to release of an active agent over time that might or might not be at a constant level. Particularly, “controlled release” encompasses situations and formulations where the active ingredient is not necessarily released at a constant rate, but can include increasing release over time, decreasing release over time, and/or constant release with one or more periods of increased release, decreased release, or combinations thereof. Thus, while “sustained release” is a form of “controlled release”, the latter also includes delivery modalities that employ changes in the amount of an active agent that are delivered at different times.

In some embodiments, the sustained release formulation, the controlled release formulation, or the combination thereof is selected from the group consisting of an oral formulation, a peroral formulation, a buccal formulation, an enteral formulation, a pulmonary formulation, a rectal formulation, a vaginal formulation, a nasal formulation, a lingual formulation, a sublingual formulation, an intravenous formulation, an intraarterial formulation, an intracardial formulation, an intramuscular formulation, an intraperitoneal formulation, a transdermal formulation, an intracranial formulation, an intracutaneous formulation, a subcutaneous formulation, an aerosolized formulation, an ocular formulation, an implantable formulation, a depot injection formulation, a transdermal formulation and combinations thereof. In some embodiments, the route of administration is selected from the group consisting of oral, peroral, buccal, enteral, pulmonary, rectal, vaginal, nasal, lingual, sublingual, intravenous, intraarterial, intracardial, intramuscular, intraperitoneal, transdermal, intracranial, intracutaneous, subcutaneous, ocular, via an implant, and via a depot injection. Where applicable, continuous infusion can enhance drug accumulation at a target site (see, e.g., U.S. Pat. No. 6,180,082). See also U.S. Pat. Nos. 3,598,122; 5,016,652; 5,935,975; 6,106,856; 6,162,459; 6,495,605; and 6,582,724; and U.S. Patent Application Publication No. 2006/0188558 for transdermal formulations and methods of delivery of compositions. In some embodiments, the administering is via a route selected from the group consisting of peroral, intravenous, intraperitoneal, inhalation, and intratumoral.

The particular mode of administration of the compositions of the presently disclosed subject matter used in accordance with the methods disclosed herein can depend on various factors, including but not limited to the formulation employed, the severity of the condition to be treated, whether the active agents in the compositions (e.g., an anti-fibrotic) are intended to act locally or systemically, and mechanisms for metabolism or removal of the active agents following administration.

### V. Methods of Using the Presently Disclosed Compositions

Based on the targeting activity of the peptides of the presently disclosed subject matter, compositions that comprise the disclosed peptides can be employed for various uses.

In some embodiments, the compositions and methods of the presently disclosed subject matter can be used for treating and/or preventing fibrosis. In such embodiments, the presently disclosed methods comprise, consist essentially of, or consist of administering to a subject in need thereof a therapeutically effective amount of a composition as disclosed herein. In some embodiments, the composition is a liposomal composition that comprises a liposome conjugated to a peptide and encompassing an anti-fibrotic agent. In some embodiments, the peptide is MDLSLKP (SEQ ID NO: 19) and the liposome targets a cell expressing collagen IIIa and/or the extracellular matrix (ECM) that is present in the subject to deliver the anti-fibrotic agent thereto.

In some embodiments, the cell is present in the pancreas of the subject and the subject is at risk for developing chronic pancreatitis (CP) and/or pancreatic fibrosis. Delivery of the anti-fibrotic, in some embodiments in combination with one or more other therapeutically effective agents in a combination therapy, reduces and/or prevents the formation of fibrosis to thereby treat and/or prevent fibrosis in the subject.

As is known in the field of medicine, chronic pancreatitis (CP) and/or pancreatic fibrosis can be associated with other diseases, disorders, or conditions that result or are otherwise sequelae of the CP or pancreatic fibrosis. Accordingly, in some embodiments the presently disclosed subject matter relates to methods for decreasing the incidence of a disease, disorder, or condition associated with chronic pancreatitis (CP), the method comprising administering to a subject in need thereof a therapeutically effective amount of a composition (e.g., a liposomal compositions) as disclosed herein. In some embodiments, the composition comprises a peptide comprising the amino acid sequence MDLSLKP (SEQ ID NO: 19), and the composition (e.g., the liposome) targets a cell expressing collagen IIIa that is present in the subject. In some embodiments, the disease, disorder, and/or condition associated with CP and/or pancreatic fibrosis is selected from the group consisting of pulmonary disease, diabetes mellitus, and pancreatic cancer.

In some embodiments, the antifibrotic agents in apigenin. The administration of apigenin can be associated with side effects, including but not limited to hepatotoxicity. Therefore, in some embodiments the presently disclosed subject matter relates to methods for decreasing the incidence of a side effect associated with apigenin treatment in a subject, including but not limited to hepatotoxicity. In some embodiments, the relevant methods comprise, consist essentially of, or consist of administering to the subject a delivery vehicle (e.g., a liposome(comprising a peptide selected from the group consisting of SEQ ID NOs: 3-38, and further wherein the delivery vehicle (e.g., liposome) encapsulates the apigenin. While not wishing to be bound by any particular theory of operation, the encapsulation of the apigenin by the delivery vehicle is believed to shield various sensitive targets from the apigenin, thereby reducing the undesirable side effects experiences by the subject.

Any subject for whom apigenin treatment might be desirable can be administered apigenin encapsulated by a delivery vehicle of the presently disclosed subject matter in order to reduce the incidence of side effects associated with apigenin treatment, including but not limited to apigenin-related hepatotoxicity. An exemplary subject for whom apigenin treatment might be desirable is a subject that has or is at risk of developing chronic pancreatitis (CP) and/or a disease, disorder, and/or condition associated with CP.

Additional peptides of the presently disclosed subject matter can also be employed as targeting agents in the method of the presently disclosed subject matter. Thus, in some embodiments the presently disclosed subject matter relates to methods for targeting active agents to various targets, which methods comprise, consist essentially of, or consist of contacting the target with a composition (e.g., a liposomal composition) as disclosed herein. In some embodiments, the target is selected from the group consisting of an acinar cell, an activated pancreatic stellate cell (aPSC), a component of the extracellular matrix (ECM), and a macrophage. In some embodiments, the composition comprises a peptide comprising, consisting essentially of, or consisting of an amino acid sequence as set forth in any one of SEQ ID NOs: 14, 19, 20, 27, and 28 in order to deliver active agents of interest to the acinar cell, the activated pancreatic stellate cell (aPSC), the component of the extracellular matrix (ECM), and/or the macrophage.

### EXAMPLES

The presently disclosed subject matter will be now be described more fully hereinafter with reference to the accompanying EXAMPLES, in which representative embodiments of the presently disclosed subject matter are shown. The presently disclosed subject matter can, however, be embodied in different forms and should not be construed as limited to the embodiments set forth herein. Rather, these embodiments are provided so that this disclosure will be thorough and complete, and will fully convey the scope of the presently disclosed subject matter to those skilled in the art.

### Materials and Methods for the Examples

Lipids for liposome preparation. 1,2-Dioleoyl-sn-glycerol-3-phosphocholine (DOPC), and 1,2-distearoyl-sn-glycero-3-phosphoethanolamine-N-[methoxy(polyethylene glycol)-2000] (DSPE-PEG₂₀₀₀) were purchased from Avanti Polar Lipids, Alabaster, Alabama, United States of America; DSPE-PEG₃₄₀₀-maleimide was purchased from Laysan Bio Inc., Arab, Alabama, United States of America; 1,1′-dioctadecyl-3,3,3′,3′-tetramethylindodicarbocyanine, 4-chlorobenzenesulfonate salt (DiD) was purchased from Biotium Inc., Hayward, California, United States of America; cholesterol was purchased from MilliporeSigma, Burlington, Massachusetts, United States of America. Peptides were synthesized by the Tufts University Peptide Synthesis Core Facility using standard FMOC chemistry and Rink-Amide resin (Tufts University, Boston, Massachusetts, United States of America). Caerulein was purchased from Bachem, Torrance, California, United States of America. Apigenin was purchased from MilliporeSigma, St. Louis, Missouri, United States of America.

Fluorophores for ex vivo imaging and antibodies for immunostaining. Fluorophore VIVOTAG® 645 (VT645), VIVOTAG® 680 (VT680), and VIVOTAG® S-750 (VT750) were purchased from PerkinElmer, Waltham, Massachusetts, United States of America, to guide tracking of phage clones by ex vivo imaging systems. Antibodies used for immunofluorescence are rat anti-mouse CD31 at 1:200 dilution (endothelial markers; BD Biosciences, San Jose, California, United States of America), rat anti-mouse CD206 at 1:1000 dilution (M2 macrophage markers; Bio-Rad, Hercules, California, United States of America), rabbit anti-mouse cytokeratin 7 at 1:3000 dilution (CK-7, epithelial markers; Abcam, Cambridge, Massachusetts, United States of America), rabbit anti-mouse collagen IIIa at 1:200 dilution (ECM markers, Abcam, Cambridge, Massachusetts, United States of America), goat anti-mouse carboxypeptidase A1 at 1:200 dilution (CPA1, acini markers; R&D Systems, Inc., Minneapolis, MN), rat anti-mouse F4/80 at 1:500 (macrophage markers; Bio-Rad, Hercules, California, United States of America), and FITC-conjugated, mouse anti-mouse α-SMA at 1:200 dilution (MilliporeSigma, St. Louis, Missouri, United States of America). Secondary antibodies containing AF488 that were used to identify the primary antibodies are donkey anti-goat at 1:500, donkey anti-rat at 1:500, and donkey anti-rabbit at 1:500 (Abcam, Cambridge, Massachusetts, United States of America). Antibodies used for western blots are rabbit anti-mouse SOD1 at 1:1000 dilution (Cell Signaling, Danvers, Massachusetts, United States of America), rabbit anti-mouse HSP70 at 1:1000 dilution (Cell Signaling, Danvers, Massachusetts, United States of America), and mouse anti-mouse β-actin at 1:1000 (Cell Signaling, Danvers, MA). The antibody used for immunohistochemistry is anti-fibronectin at 1:50 dilution (Abcam, Cambridge, Massachusetts, United States of America).

Caerulein-induced pancreatitis in mice. All animal experiments were approved by the Animal Care and Use Committee at the University of Virginia, Charlottesville, Virginia, United States of America and conformed to the United States National Institutes of Health “Guide for the Care and Use of Laboratory Animals in Research.”

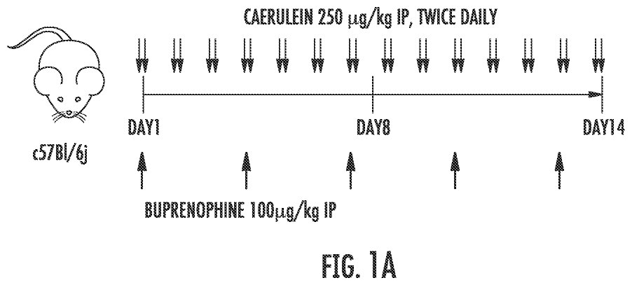

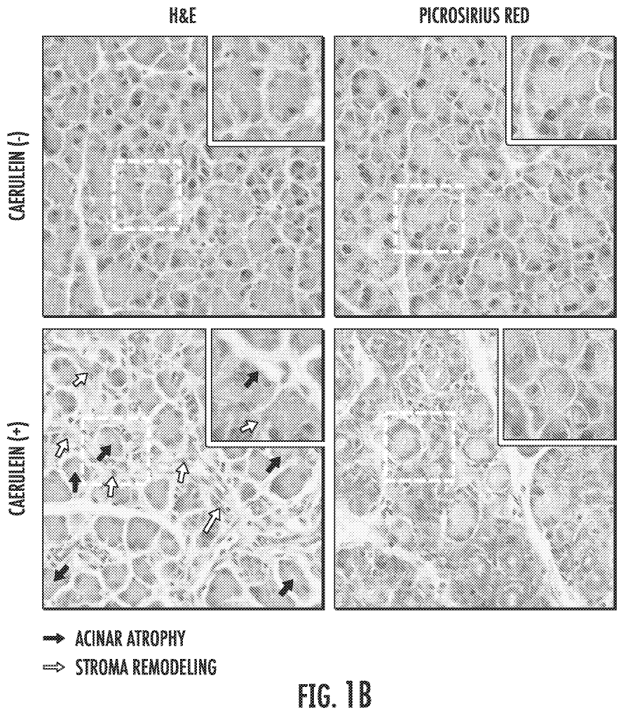

C57BL/6 J mice (6-12 week-old, female) were used for the in vivo phage screening. For chronic pancreatitis, caerulein (Bachem, Torrance, California, United States of America) was dissolved in sterile saline and administrated to mice twice a day, 8 hours apart for 14 consecutive days, at a concentration of 250 μg/kg body weight via intraperitoneal injection (Halbrook et al., 2017), as shown in FIG. 1A. During the 14-day course, intraperitoneal injections of 100 μg/kg buprenorphine were given every 3 days to minimize the induced pain. For healthy controls, c57BL/6 J mice were injected intraperitoneally with an equal volume of sterile saline following the same schedule as in the chronic pancreatitis models. The inflammatory status of the pancreas was confirmed at the end of caerulein treatments via immunohistochemistry using hematoxylin-eosin (H&E) and picrosirius red (Polysciences, Inc., Warrington, Pennsylvania, United States of America) staining (FIG. 1B). Picrosirius red (Polysciences, Inc., Warrington, Pennsylvania, United States of America) was used to visualize collagen in paraffin-embedded pancreas sections (FIG. 1B).

In vivo phage screening. 1×10¹² plaque-forming units (pfu) of the PH.D.™-7 Phage Display Peptide Library (New England Biolabs, Ipswich, Massachusetts, United States of America) were injected intravenously via tail vein into CP and healthy C57BL/6J mice (n=3, each). Phage were allowed to circulate in the blood system for 4 hours post-injection to allow extravasation out of the bloodstream and into tissues to facilitate cellular binding before the mice were euthanized. Various organs including heart, liver, spleen, pancreas, kidneys, and skeletal muscle, were harvested, weighed, and homogenized in a lysis buffer: 1× EDTA and 1× protease inhibitor cocktail (Fisher Scientific, Hampton, New Hampshire, United States of America) in Dulbecco's phosphate-buffered saline (DPBS, HyClone, Logan, Utah, United States of America). Phage titers of the tissues were determined by bacteriophage plaque assay following the manufacturer's instructions (PH.D.™ Phage Display Libraries Instruction Manual, New England Biolabs, Ipswich, Massachusetts, United States of America) and calculated in percent injected dose (% ID) per organ-weight. Phage recovered from the pancreas pool were amplified in Escherichia coli strain ER2738 at the early-log phase in LB media for 5 hours at 37° C. Bacterial debris was centrifuged at 12,000 rpm for 10 minutes, and phage in the supernatant were purified via PEG precipitation (PEG/NaCl: 20% w/v polyethylene glycol-8000, 2.5 mM NaCl) overnight at 4° C. The amplified phage were then washed with DPBS, precipitated again with PEG/NaCl at 4° C. for 30 minutes, centrifuged, and resuspended in DPBS for the next round of biopanning. Three rounds of biopanning were performed in both CP and healthy mice.

Phage DNA sequencing. Thirty phage plaques from the pancreas of round-3 were randomly selected for DNA sequencing. The insert oligo in the integrated section of the phage was amplified by polymerase chain reaction (PCR) using a forward primer having the nucleotide sequence 5′-CCTTTAGTGGTACCTTTCTAT-3′ (SEQ ID NO: 1) and a reverse primer having the nucleotide sequence 5′-GCCCTCATAGTTAGCGTAACG-3′ (SEQ ID NO: 2), and then Sanger sequenced (Eurofins). For next-generation sequencing, phage DNA of the pancreas pooled at all rounds was extracted using sodium iodide precipitation, followed by PCR amplification using a forward primer with the sequence AATGATACGGCGACCACCGAGATCTACACTCTTTCCCTACACGACGCTCTTCC GATCTTTATTCGCAATTCCTTTAGTGG (SEQ ID NO: 39) in combination with one or more reverse primers selected from the group consisting of SEQ ID NOs: 40-63 (see also Brinton et al., 2016) with KAPA HiFi PCR kit (Fisher Scientific, Hampton, New Hampshire, United States of America). The PCR cycles are initialized with one step of 95° C. for 1 minute, followed by 17 cycles of 95° C. for 30 seconds, 60° C. for 30 seconds, and 72° C. for 30 seconds. PCR purification was performed using a QIAQUICK® PCR purification kit (Qiagen, Hilden, Germany) following the manufacturer's instructions. Phage DNA was sent to the UVA Biomolecular Research Core Facility for single-ended Next-generation sequencing (NGS) on an Illumina Miseq Sequencer. FASTA files generated from the Illumina sequencing were processed by PHASTpep software previously described in Brinton et al., 2016. In brief, PHASTpep recognizes and translates the inserted DNA sequences into 7-amino acids peptides. Combining with the reads obtained from NGS of the NEB naïve PH.D.™ library used for screening, PHASTpep generates matrices of ranked, normalized read frequencies, and the corresponding sequences.

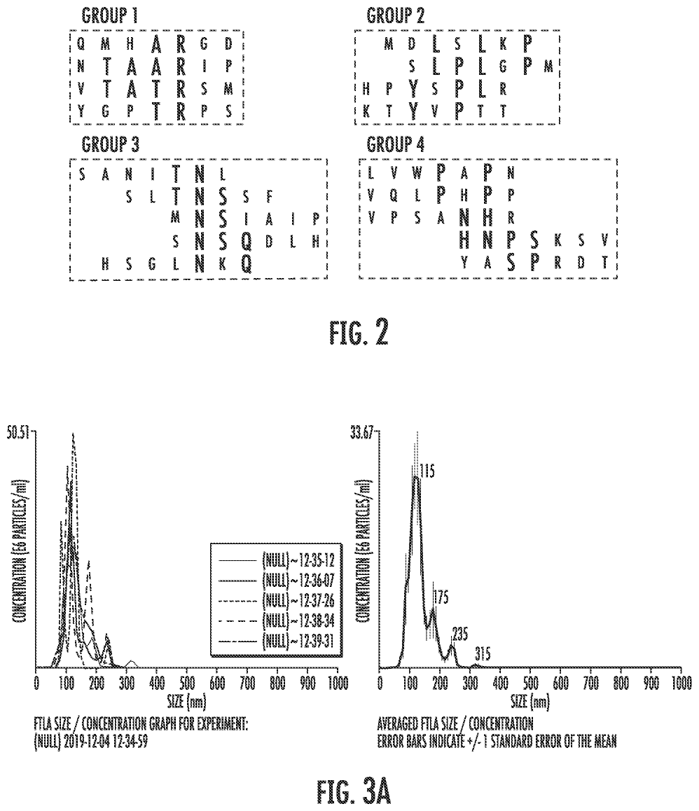

Specificity of phage clones. After PHASTpep analysis and candidate clone selections, 18 candidate clones were grouped (4-5 clones per group, 4 groups total) based on sequence similarity using GibbsCluster Server 2.0 (Andreatta et al., 2017). by the Technical University of Denmark (FIG. 2). Phage clones were pooled in equimolar amounts and labeled with fluorophore VIVOTAG® 645 (VT645, PerkinElmer, Waltham, Massachusetts, United States of America). Wild type M13Ke phage, used as the negative control to account for phage background bindings, was labeled with VIVOTAG® S-750 (VT750, PerkinElmer, Waltham, Massachusetts, United States of America). For each group, the fluorescently labeled candidate and WT phage were mixed and co-injected in CP and healthy mice (n=5) Phage accumulation was determined by measuring the ex vivo fluorescent intensity of the pancreas at 20 hours post-injection using the Fluorescent Molecular Tomography (FMT, PerkinElmer, Waltham, Massachusetts, United States of America) using the excitation and emission wavelength at 675 nm/720 nm for VT680 and 745 nm/800 nm for VT750. To account for variations of fluorescent labeling efficiency, the raw readouts of VT680 and VT750 were normalized to the dye-per-phage ratio (see Table 3 below).

The specificity ratio was calculated as % ID/g of the normalized VT680 divided by the % ID/g of the normalized VT750 (see Equation (1)). Quantifications are included in Table 4.

$$\% ID / g = \frac{dye in pancreas}{phage infected \times dye / phage} \times \frac{1}{{mass}_{panc}} \times 100 \% \qquad Equation 1$$

A similar procedure was applied to determine the specificity of individual phage clones in Groups 2 and 3. Candidate clones were labeled with VivioTag 680 (VT680, PerkinElmer, Waltham, MA) to match the detectable wavelength of the IVIS Spectrum Series (Perkin Elmer, Waltham, Massachusetts, United States of America). Fluorescent intensity was measured at 20 hours post-injection of candidate phage (VT680) and WT phage (VT750). Fold change of specificity ratio was calculated as the specificity ratio of CP mice divided by the specificity ratio of the healthy mouse (see Table 5).

Immunofluorescence (IF). Post ex vivo imaging, mouse pancreata were submerged in Neg-50 Frozen Section Medium (Thermo Scientific, Waltham, Massachusetts, United States of America) and snap-frozen over liquid nitrogen vapor. The embedded tissues were cut into 5 μm sections using a cryostat (Leica Microsystems Inc., Buffalo Grove, Illinois, United States of America) for subsequent imaging with ZEISS LSM-880 Confocal Laser Scanning Microscope (Carl Zeiss Meditec, Inc., Jena, Germany) at the Advanced Microscopy Facility at the University of Virginia. Cell types of interest in the pancreatitis microenvironment were identified by immunohistochemistry using the following antibodies: rat anti-mouse CD31 at 1:200 dilution (endothelial markers; BD Biosciences, San Jose, California, United States of America), rat anti-mouse CD206 at 1:1000 dilution (M2 macrophage markers; Bio-Rad, Hercules, California, United States of America), rabbit anti-mouse cytokeratin 7 at 1:3000 dilution (CK-7, epithelial markers; Abcam, Cambridge, Massachusetts, United States of America), rabbit anti-mouse collagen IIIa at 1:200 dilution (ECM markers; Abcam, Cambridge, Massachusetts, United States of America), goat anti-mouse carboxypeptidase A1 at 1:200 dilution (CPA1, acini markers; R&D Systems, Inc., Minneapolis, Minnesota, United States of America), rat anti-mouse F4/80 at 1:500 (macrophage markers; Bio-Rad, Hercules, California, United States of America), and FITC-conjugated, mouse anti-mouse α-SMA at 1:200 dilution (MilliporeSigma, St. Louis, Missouri, United States of America). ALEXA FLUOR® 488 (Abcam, Cambridge, Massachusetts, United States of America) antibodies from appropriate species, including donkey anti-goat, donkey anti-rat, donkey anti-rabbit at 1:500 dilution, were used as secondary antibodies to locate the primary antibodies. IF-stained pancreatic sections were mounted with PROLONG™ Gold Antifade Mountant with DAPI (Thermo Fisher Scientific Inc, Waltham, Massachusetts, United States of America, United States of America) for nucleus visualization. IF images were taken on Ziess LSM 880 Confocal Microscope (ZIESS, Oberkochen, Germany) at the Advanced Microscopy Facility at the University of Virginia.

Preparation and characterization of liposomes. Peptide 7-mers identified in the phage screen were chemically synthesized including a C terminal addition of the amino acid linker, GGSKC (SEQ ID NO: 4) by the Tufts University Peptide Synthesis Core Facility using standard FMOC chemistry and Rink-Amide resin The lysine at position 4 of the linker was conjugated to a 5-carboxyfluorescein (5-FAM) fluorophore. Exemplary such peptides included the amino acid sequences MDLSLKPGGSKC (SEQ ID NO: 64), wherein the lysine at position 11 was conjugated to a 5-FAM fluorophore, and MNSIAIPGGSKC (SEQ ID NO: 65), wherein the lysine at position 11 was conjugated to a 5-FAM fluorophore. Liposome preparation was carried out as previously described with minor modifications (Dasa et al., 2015). In brief, 4 mg of peptides were first conjugated to 9.5 mg of DSPE-PEG₃₄₀₀-maleimide in 1 mL of 0.5 mM EDTA/PBS under argon to prepare the aqueous micellar solution. The micelle mixture was left for 1 hour at room temperature, followed by overnight incubation at 4° C. Overnight dialysis was performed in PBS and then in MilliQ H₂O twice to remove free peptides and salts from the conjugated micelles. The purified

DSPE-PEG₃₄₀₀-peptide was then lyophilized and ready for use in liposome preparations. Liposomes were prepared by hydration of lipid film composed of the following reagents: DOPC (9.5 mg), cholesterol (4.5 mg), DSPE-PEG₂₀₀₀ (4.5 mg), DSPE-PEG₃₄₀₀-peptide (1 mg), and DiD) (0.5 mg). DiD was incorporated into the lipid bilayer as a non-exchangeable near-infrared lipid dye, allowing in vivo detection of liposomes by IVIS (PerkinElmer,

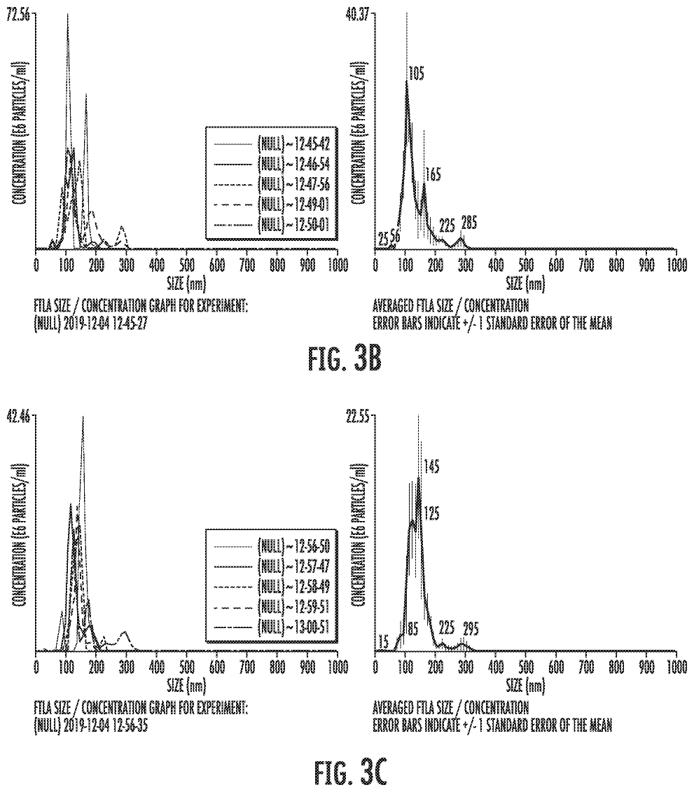

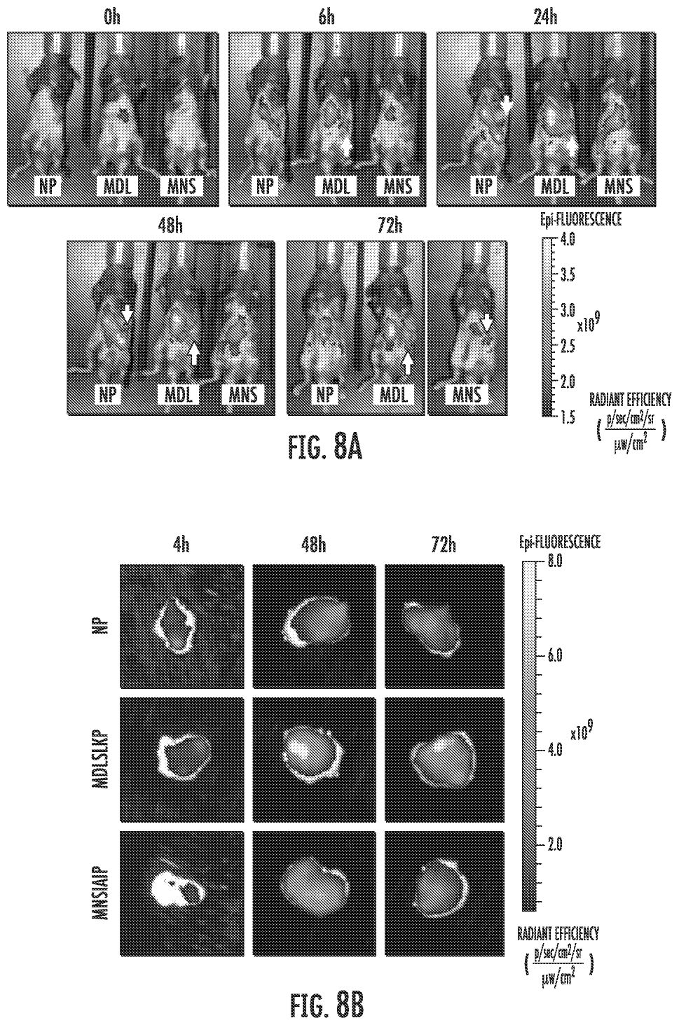

Waltham, Massachusetts, United States of America). The lipid contents were mixed by sonication in 1 mL of chloroform, 1 mL of saline, and 3 mL of ether followed by placing on a rotary evaporator overnight to remove residual organic solvents. The lipid mixtures were then size-extruded 21 times through a syringe extruder with a 0.2 μm Nuclepore filter (Thermo Fisher Scientific, Inc., Waltham, Massachusetts, United States of America). The size-extruded liposomes were centrifuged on A-100/18 Fixed-Angle Rotor (Beckman Coulter, Brea, California, United States of America) by AIRFUGER Air-Driven Ultracentrifuge (Beckman Coulter, Brea, California, United States of America) at 20 pounds per square inch gauge (psig) for 1 hour to separate micelles and unattached lipids from liposomes. The resulting liposomal pellets were resuspended in saline and characterized by NanoSight NS300 (Malvern Instruments Ltd., Worcestershire, United Kingdom) to determine particle size and concentration (FIGS. 3A-3C and 8A-8C). The settings of NanoSight are as follows, camera level=11; slider shutter=600; slider gain=300; FPS =25.0; number of frames=749; temperature=20.7-20.9° C.; viscosity=(water) 0.979-0.982 cP. The absorbance of FAM at 495 nm was used to determine the number of peptides incorporated in each liposome formulation.

Specificity of peptide-conjugated liposomes. Peptide-conjugated liposomes (150 μL containing 5×10¹¹ particles) were injected via tail vein in CP mice (n=3) to determine the pharmacokinetic properties using IVIS. No peptide liposomes were used as negative controls to account for background bindings of liposomes. Mice hair were shaved and removed by depilatory creams prior to imaging. In vivo imaging on IVIS at 0, 6, 24, 48 and 72 hours post injections was performed. 4, 48, and 72 hours post injections, animals were perfused with saline, and organs were harvested for ex vivo imaging on IVIS using the Ex/Em 640/680 nm filter sets to detect DiD accumulation in the pancreas and other organs. The cellular targets of targeting liposomes were determined by IF using antibodies against the acinar cell marker (CPA1), aPSC marker (αSMA), ECM marker (collagen IIIa), epithelial marker (CK7), endothelial marker (CD31), and macrophage marker (F4/80). Co-localization analysis was done on the JACOP plugin of the ImageJ software (National Institutes of Health, Bethesda, Maryland, United States of America; see also Abramoff et al., 2004; Schneider et al., 2012). Mander's co-localization coefficient (MCC), which represents the percentage of liposomes overlapping with cell markers, was used as an indicator to quantitate the extent of co-localization of liposomes with each cell type (Manders et al., 1993).

Apigenin drug loading and release kinetics. The lipid mixture, containing DOPC (9.5 mg), cholesterol (4.5 mg), DSPE-PEG₂₀₀₀ (4.5 mg), DSPE-PEG₃₄₀₀-peptide (1 mg), and 2 mg apigenin were pre-dissolved in 100 μL ethanol, respectively. The drug and lipid mixture were mixed and added to 1 mL PBS at 55ºC for 1 hour. Liposomes were prepared by passing through a 0.2 μm Nucleopore filter using a syringe extruder. The free drug was removed by Zeba Spin Desalting Column, 40K molecular weight cut off (MWCO; Thermo Scientific, Waltham, Massachusetts, United States of America), which was pre-washed three times with saline. The resulting liposomes were characterized by NanoSight NS300. The drug loading per liposome was determined by Ultrospec 3000 UV/visible spectrophotometer (Pharmacia Biotech, Uppsala, Sweden). The extinction coefficient of apigenin in saline is 664.02 (M⁻¹cm⁻¹) at 337 nm, which was determined using Beer's law on serial-diluted samples of known concentrations. To determine the degree of apigenin maintained encapsulated in the purified liposomes at storage condition (PBS, 4° C.), a release kinetic study was performed for 14 days. The purified liposomes were spun down in Pierce Protein Concentrators PES, 10K MWCO (Thermo Scientific, Waltham, Massachusetts, United States of America) on day 0, 9, and 14 at storage conditions. The free drug concentration in the filtrate was determined by UV spectrometer to calculate amount of free apigenin released from the purified liposomes. In vitro release kinetics was determined by placing drug-loaded liposomes in the cartridge of SLIDE-A-LYZER™ Mini Dialysis devices, 10K MWCO (Thermo Fisher Scientific Inc, Waltham, Massachusetts, United States of America), with the conical tube filled with 50% FBS/saline at 37° C. The FBS buffer was used as a blank to set up UV spectrometer readouts. Free drug released into FBS buffer were collected at day 0, 1, 3, and 4 post incubation and determined by UV spectrometer.

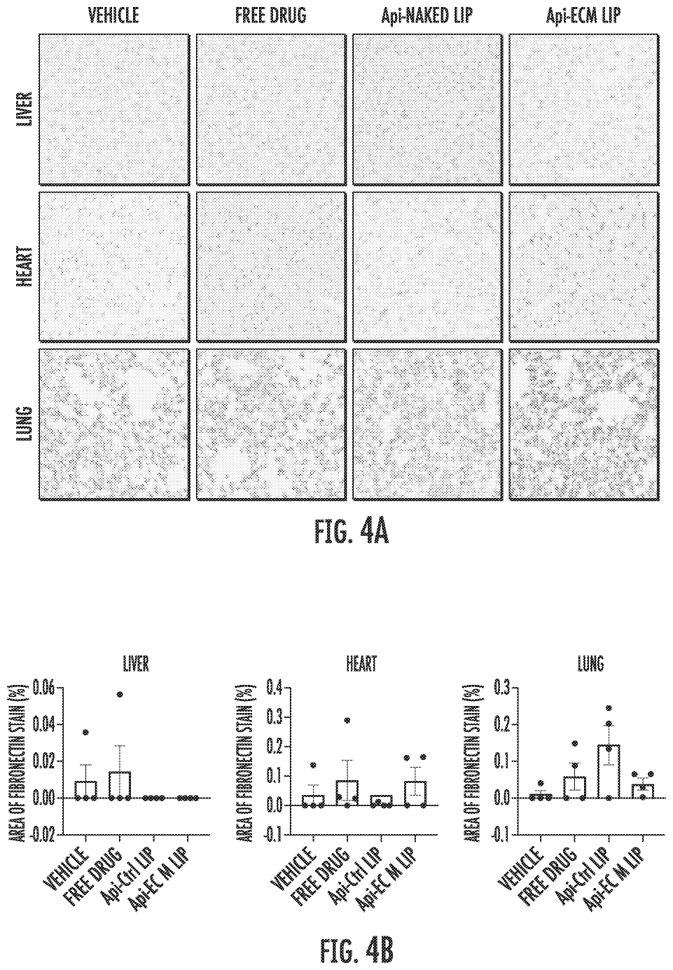

Pharmacodynamic measurement and evaluation of therapeutic efficacy of targeted delivery of apigenin. 6 weeks old C57BL/6J mice were given intraperitoneal injections of caerulein (125 μg/kg, twice daily, 5 weeks). Following 2 weeks of caerulein treatment, animals were randomly divided into 4 groups, n=5 per group: (1) vehicle - intravenous injections of MDLSLKP (SEQ ID NO: 19) conjugated liposomes (ECM Lip), twice-weekly; (2) free drug—oral gavage of apigenin, 2 mg/kg, 6 days per week (Mrazek et al., 2015); (3) Api-NP Lip—intravenous injections of apigenin encapsulated no peptide liposomes, 6 mg/kg, twice per week; and (4) Api-ECM Lip—intravenous injections of apigenin encapsulated, MDLSLKP (SEQ ID NO: 19) conjugated liposomes, 6 mg/kg, twice per week. Apigenin treatments lasted for 3 weeks for the remaining 3 weeks of caerulein induction. At the end of week 5, mice were euthanatized and serum harvested through cardiac puncture. Pancreata were perfused, harvested, and paraffin-embedded for immunohistochemistry staining. Livers were perfused, harvested, and partially paraffin-embedded for histology staining. The rest of the liver tissues was homogenized in the lysis buffer (200 mM HEPES, pH 7.5, 10 mM KCl, 1.5 mM MgCl₂, 1 mM EDTA, 1 mM EGTA, 2 mM PMSF, 1 mM DTT, and 1× Protease Inhibitor Cocktail (Thermo Fisher Scientific Inc, Waltham, Massachusetts, United States of America; Singh et al., 2012). Pancreata sectioned at 5 μm were stained with H&E, picrosirius red, and fibronectin (1:50, Abcam, Cambridge, Massachusetts, United States of America). Visualization of fibronectin was done with 3′3-diaminobenzidine (DAB; Acros Organics, Fair Lawn, New Jersey, United States of America) with counter staining using Hematoxylin 1 (Richard Allen Scientific, San Diego, California, United States of America). The quantification of picrosirius red was determined by Image J, using threshold applied on the red composites of the RGB images. In the analysis, a total of 60 images per group (12 images per animal) were used for picrosirius red quantification. Fibronectin expression was quantified using the positive pixel count function on QuPath software (Bankhead et al., 2017). Acinar atrophy was determined by counting the number of damaged acinus within a 256 μm×256 μm region of interest in the pancreas H&E images. 5 images/animal, 3 animals/group were analyzed. Hepatoxicity induced by the treatments was evaluated by western blot of the liver lysates probing against superoxide dismutase 1 (SOD1; rabbit anti-mouse 1:1000, Cell Signaling, Danvers, Massachusetts, United States of America) and B-actin (mouse anti-mouse 1:1000, Cell Signaling, Danvers, Massachusetts, United States of America). Serum alanine aminotransferase (ALT) and blood urea nitrogen (BUN) were determined on VetTest 8008 Chemistry Analyzer (IDEXX, Westbrook, Maine, United States of America). Liver, heart, and lung from all treatment groups were sectioned and stained with fibronectin to evaluate treatments in tissues other than the pancreas (FIGS. 4A and 4B).

Statistical analysis. Statistical analysis of the data was performed by student t-test, one-way analysis of variance (ANOVA), and Tukey-Kramer test. All data presented are expressed as mean+standard error of at least three independent measurements. For all comparisons, p-values<0.05 were considered significant.

### EXAMPLE 1

### Enrichment of Phage Clones Specific to the Inflamed Pancreas

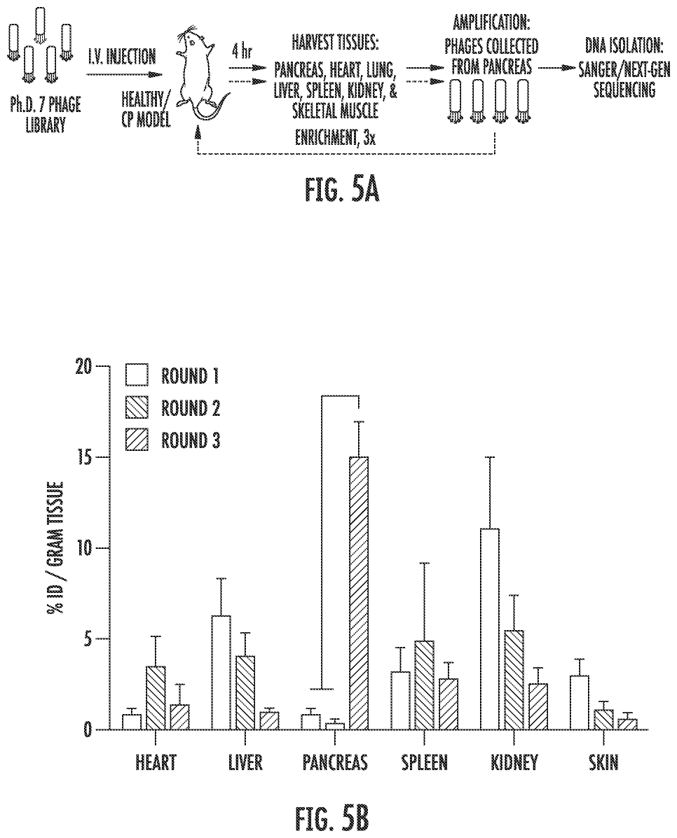

The present co-inventors have pioneered methods to identify novel ligands and targets for various diseases (Bausch et al., 2011, Dasa et al., 2015; Brinton et al., 2016; Gutknecht et al., 2017). To select phage clones that bind specifically to cells in the complex CP microenvironment, an in vivo phage display screen was performed by injecting the phage library into the caerulein-induced CP mouse model (FIG. 5A). The PH.D.™7 phage library (1×10¹² pfu per mouse) was injected via tail vein in CP mice and allowed to circulate for 4 hours before animals were euthanized to allow extravasation out of the bloodstream and into tissues to facilitate cellular binding. Phage harvested from the pancreas were amplified and re-injected into CP animals for a total of 3 rounds of biopanning. For each round, tissue other than pancreas were also harvested and phage titered to determine selectivity of the phage tool for CP pancreas. After three rounds of selection, the phage titer in the CP pancreas showed a statistically significant increase from round 1 (0.89% ID/g) and round 2 (0.39% ID/g) to round 3 (14.38% ID/g; FIG. 5B). Phage titers in the clearance organs, including liver, spleen, and kidneys, decreased over rounds, implying the selectivity of phage pools shifted towards the inflamed tissues over the enrichment process as expected for a successful enrichment process. At the end of round 3, phage clones isolated from pancreata were Sanger and deep sequenced (30 clones per animal; n=3) to identify CP-specific peptides using an in silico selection approach from the enriched phage pools

### EXAMPLE 2

### In Silico Selection of CP-specific Candidate Clones

The Phage Analysis for Selective Targeted PEPtides (PHASTpep) software has been previously demonstrated to identify target-specific phage clones in screening against recombinant proteins and cells in culture (Brinton et al., 2016). The process involves phage display selection, then Next-Generation Sequencing (NGS) to identify the peptide sequences that were able to bind the target. From there, peptide sequences are subjected to the selectivity algorithm, which searches through a database containing data from all previous phage display experiments performed by the co-inventors (hereinafter “the Kelly Database”). This step functions as an in silico negative selection to remove any phage clone that appears in multiple screens and would therefore, be non-specific or non-selective and capable of binding to multiple targets. To ensure selectivity for the inflamed pancreas, in addition to the caerulein-treated mice, healthy mice of the same mouse strain were screened and the outputs from these screens were employed in the algorithms to in silico counter-select clones that also bound to the healthy pancreas. PHASTpep applied normalization algorithms to remove non-specific binders and to ensure selectivity (Brinton et al., 2016). However, the algorithms were used for cell culture selections with on cell type present. Four different methods to analyze the data and choose CP-specific peptide ligands were developed to account for a more complex system that involves thousands of available targets expressed in the multiple cell types present in an in vivo screen.

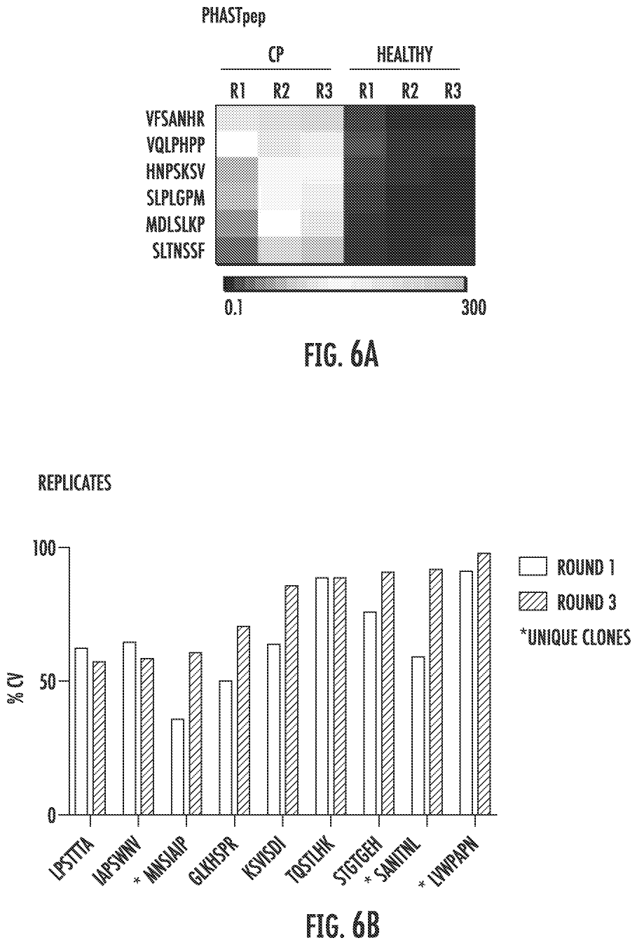

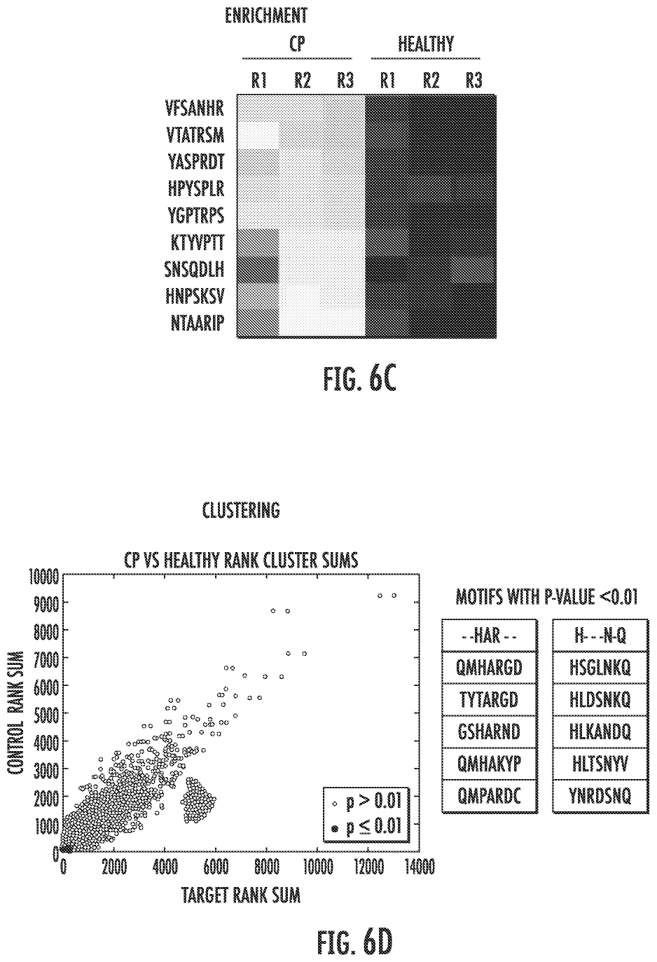

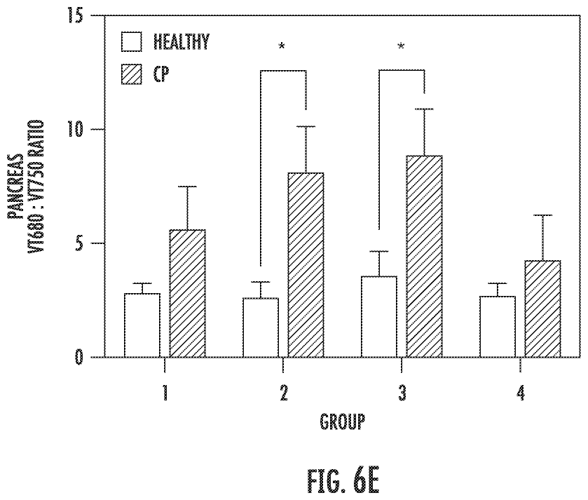

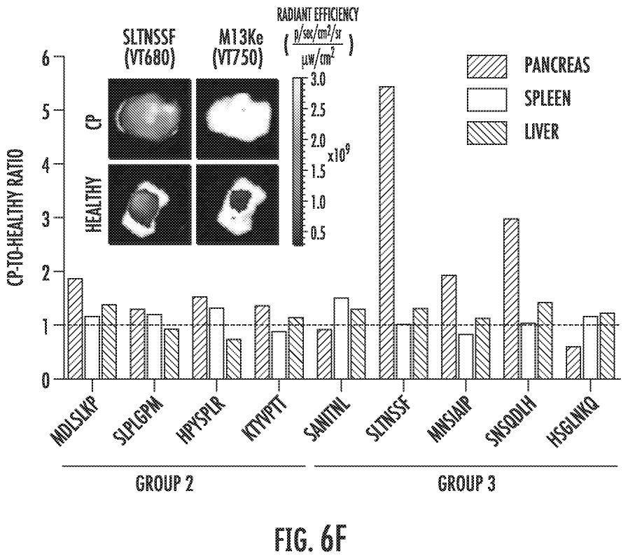

PHASTpep for clone selection. Conventional clone picking suffers from high false-positive rates and lacks a robust approach to select target-specific candidate clones. To address this issue, the NGS frequency counts from round 3 were used to allow quantitative sorting. To ensure specificity, each individual clone's frequency count in the target screens was normalized to that in the naïve library screen (normalized frequency) to account for amplification and library biases (Brinton et al., 2016). To ensure selectivity for CP pancreas over the healthy pancreas, normalized frequency of clones from CP pancreas screen were compared with pancreas from healthy animals. Of the 90 clones that Sanger sequencing methods alone would have identified, only 6 clones were selected as meeting the criteria of high normalized frequency counts in the CP pancreas (>50) but low accumulation in the healthy pancreas (<10; FIG. 6A) These selection criteria ensured a 5-10-fold higher expression of phage clone in the inflamed over benign pancreas. It also removed a non-specific phage clone, ADARYKS (SEQ ID NO: 3; Kroiss et al., 2019. See also Guo et al., 2013 and http://parts.igem.org/Part:BBa_K2043011:Design) from the candidate list, which would have been selected using Sanger sequencing alone since it was the most abundant clone among clones picked (10%).

Replicability between libraries. Lot-to-lot variations of naïve libraries may induce amino acid distribution bias and skew the screening results (Ryvkin et al., 2018). Therefore, a method to take the variability into account and ensure the utility of the data was developed in order to be able to compare the sequences from every screen. This allows better selectivity as we can rapidly remove phage clones that are present across multiple selections. To elucidate the variability, the mean and standard deviation of the normalized frequency of a clone across two lots of phage display libraries (n=3 animals) was calculated and those clones with coefficient variants (CV) <1 and that had a minimum of 5-fold increase expression in CP over healthy pancreas were selected (FIG. 6B). Of 9 clones that met these criteria, 3 unique clones, LVWPAPN (SEQ ID NO: 18), MNSIAIP (SEQ ID NO: 20), and SANITNL (SEQ ID NO: 25) were selected.

Clone enrichments over rounds. During biopanning, high affinity binders and/or clones binding to targets with high expression tend to remain in the elutes and be enriched after amplifications. This process is indicated by a clone's increasing frequencies present over rounds. As limited amounts (<0.0001%) of clones were sequenced in the traditional clone picking, the loss of potential candidate clones occurs early in the biopanning process, making direct comparisons of individual clone's frequency change between rounds not as informative. With NGS, several orders of magnitude in the quantity of sequences in the library are obtained, enabling the calculation of a more reflective growth rate for each clone in silico. Therefore, the growth rate (GR), determined by the ratio of the numbers of clones present between rounds, can be employed to select high affinity clones. In notation, GR2(3)1 =frequency in round 2(3)/ frequency in round 1. Of the populations with the top 60 frequency counts in round 2 and 3, 9 clones that showed consistent positive GR (GR₂₁ and GR₃₁ >1) were selected, ensuring the analysis was not skewed towards burst growth in a single selection (FIG. 6C). Using this method, clones that would have been discarded using conventional clone picking alone were identified.

Homologous motif identification using clustering analysis. As protein interactions are often determined by a few amino acids, motifs conferring phage binding to its target can be repeatedly seen among different clones in the same screen. Recognizing target-specific homology families can offer insights to the libraries that would have be missed when evaluating each sequence as an independent read. Therefore, we clustered phage pools using the Hobohm algorithm (Hobohm et al., 1992. See also https://metacpan.org/pod/String:Cluster::Hobohm) to reveal homologous motifs in the CP screens. The frequency ranks of the identified homologies in the CP screens were then compared to the ranks in the healthy pancreas. Two clones, QMHARGD (SEQ ID NO: 23) and HSGLNKQ (SEQ ID NO: 12), from the two statistically significant (p-value<0.01) motif families were selected based on their target selectivity and growth rate (FIG. 6D).

After combining the four (4) analysis methods and determining selectivity using the Kelly Database of phage screens that contains tissue-specific phage clones (Brinton et al., 2016), 18 candidate clones were selected for further validation.

### EXAMPLE 3

### In Vivo Validation of Phage Clones Specific to Cellular Components in the Inflamed Pancreas

To efficiently evaluate 18 candidate clones, they were divided into four (4) Groups (see FIG. 2) based on sequence similarity using GibbsCluster Server 2.0 (Andreatta et al., 2017). Phage in the selected Group and the wildtype M13Ke phage were fluorescently conjugated to VT680 and VT750, respectively, pooled then were intravenously injected via tail vein into healthy or CP mice. At 20 hours post-injection, ex vivo ratiometric imaging of targeted-to-M13Ke phage analysis revealed the higher accumulation of phage clones from Groups 2 and 3 in the CP pancreas when compared with pancreas from healthy mice (p<0.05; FIG. 6E), while phage in Groups 1 and 4 were the same between CP pancreas and healthy pancreas. Phage accumulation of all Groups in the pancreas from healthy mice were consistent across all Groups (FIG. 6E). A total number of 9 individual clones gathered from Groups 2 and 3 were subsequently screened individually for their specificity for the inflamed pancreas. Seven out of nine clones (MDLSLKP (SEQ ID NO: 19), SLPLGPM (SEQ ID NO: 26), HPYSPLR (SEQ ID NO: 11), KTYVPTT (SEQ ID NO: 14), SLTNSSF (SEQ ID NO: 27), MNSIAIP (SEQ ID NO: 20), and SNSQDLH (SEQ ID NO: 28)) showed increased specificity for CP over healthy pancreas (FIG. 6F). From the validation results, it was concluded that PHASTpep-guided selection, replicates and enrichment algorithm can reveal sequences specific for CP pancreas from an in vivo screen. That none of the clones identified from clustering analysis showed specificity to CP could be a result of the diverse available targets present in tissues, thus, increasing the difficulty to converge valid motifs using the Hobohm algorithm.

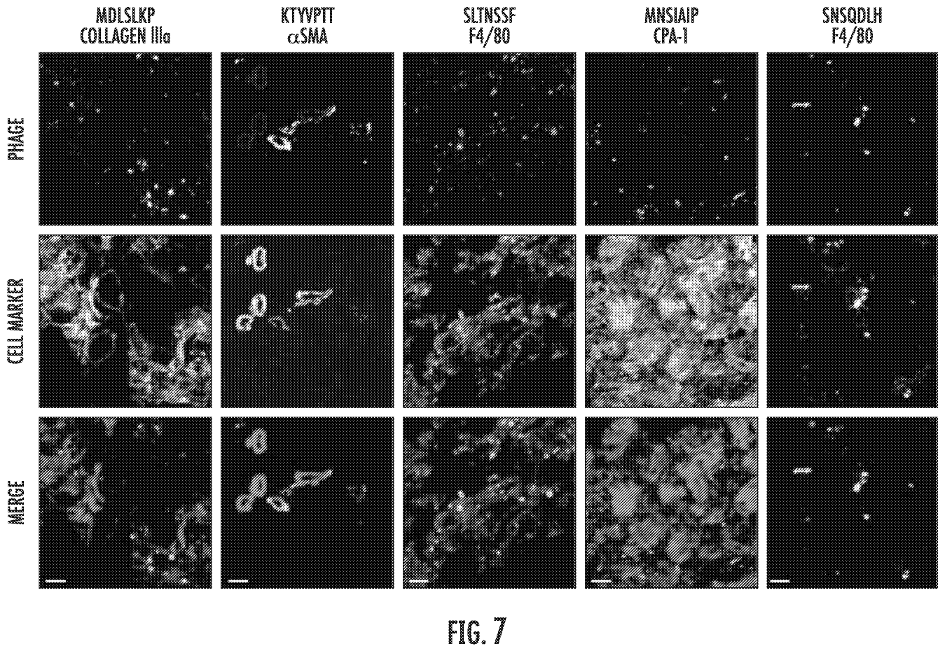

To identify the cell types that the CP targeting clones were binding, immunofluorescent analysis was performed on common cellular components in the CP microenvironment in the inflamed pancreas sections following in vivo validation of individual clones (FIG. 7). Mander's correlation coefficient (MCC) analysis was performed to determine colocalization of the targeting phage to the cell markers. Among each clone, the MCC value of each cell marker was compared to the rest of the markers using the Tukey-Kramer test (Table 6) Five clones were identified to demonstrate statistically significant colocalization to one single cell type; thus, an indication for cellular selectivity. Through this analysis, it was revealed that KTYVPTT (SEQ ID NO: 14) was selective for αSMA+ cells (MCC=0.521±0.067), MDLSLKP (SEQ ID NO: 19) for collagen IIIa+ cells (MCC=0.828±0.089), MNSIAIP (SEQ ID NO: 20) for CPA-1+ cells (MCC=0.633±0.179), and SLTNSSF (SEQ ID NO: 27) and SNSQDLH (SEQ ID NO: 28) for F4/80+ cells (MCC=0.804±0.090 and 0.800±0.197, respectively; Table 7). Phage clones and the associated cellular components in the inflamed pancreas are summarized in Table 8.

### EXAMPLE 4

### Peptide-Conjugated Liposomes Alter Nanoparticle Pharmacokinetics and Show Specificity to the Inflamed Pancreas

The extracellular matrix and acinar cells are highly abundant cells in CP, therefore peptides MDLSKLP (SEQ ID NO: 19) and MNSIAIP (SEQ ID NO: 20) were chosen to develop targeted liposomes. Despite demonstrating the highest in vivo ratio for specificity between the inflamed and healthy pancreas, we did not select the macrophage targeting peptides for this proof-of-concept drug delivery system because macrophages present in a wide spectrum of activated phenotypes in inflammatory and fibrotic diseases and are subject to change in response to microenvironmental stimuli (Stout et al., 2005; Kawanishi et al., 2010; Wynn & Barron, 2010; Hu et al., 2020; Yang et al., 2020). Better understanding the roles of macrophages in CP and further characterizations on the targeting specificity of SLTNSSF (SEQ ID NO: 27) and SNSQDLH (SEQ ID NO: 28) and subtypes of macrophage targeted is needed. Peptide-conjugated liposomes were prepared by the reverse phase evaporation method with an average size of 90-110 nm in diameters, and the number of peptides displayed on the surface ranged from 400-450 per liposome (see Table 9). All peptides employed had a net charge of +1 at pH 7.0. A non-exchangeable Lipid dye, DiD, was incorporated into the lipid formula at an average of 350-450 dye molecules per liposome to allow particle tracking by non-invasive imaging modalities (see Table 9). Liposomes without surface modifications (No-peptide liposomes; NP) were used as negative controls in the pharmacokinetics studies as these are readily taken up by the abundant phagocytic cells present in CP. 5×10¹¹ liposomes were injected into CP mice via tail vein, and the animals were imaged at 0, 6, 24, 48, and 72 hours post injection using IVIS. Starting at the 6 hour and lasted until the 72 hour timepoint, fluorescent accumulation observed in the area of the left abdomen was consistent with the location of the pancreas in the MDLSLKP (SEQ ID NO: 19) liposome-injected mice (FIG. 8A). At 48 hours post injection, a 1.3-fold increase of MDLSLKP (SEQ ID NO: 19) liposomal accumulation was detected compared to the NP liposomes (FIGS. 8B and 8C). As expected, liposome accumulation was also observed in the clearance organs (liver, spleen, and kidney) as previously reported (see Koning et al., 2002; Immordino et al., 2006). A significant reduction in liver and kidney accumulation of the MDLSLKP (SEQ ID NO: 19) liposomes was observed, suggesting the addition of targeting ligands altered the particle distribution away from the clearance organs to the inflamed pancreas. In contrast to MDLSLKP (SEQ ID NO: 19) targeted liposomes, there were not significant differences in pancreas and liver accumulations of the MNSIAIP (SEQ ID NO: 20) liposome compared to the NP liposome.

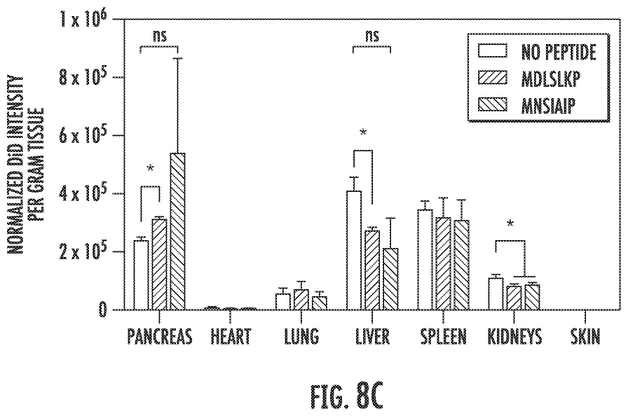

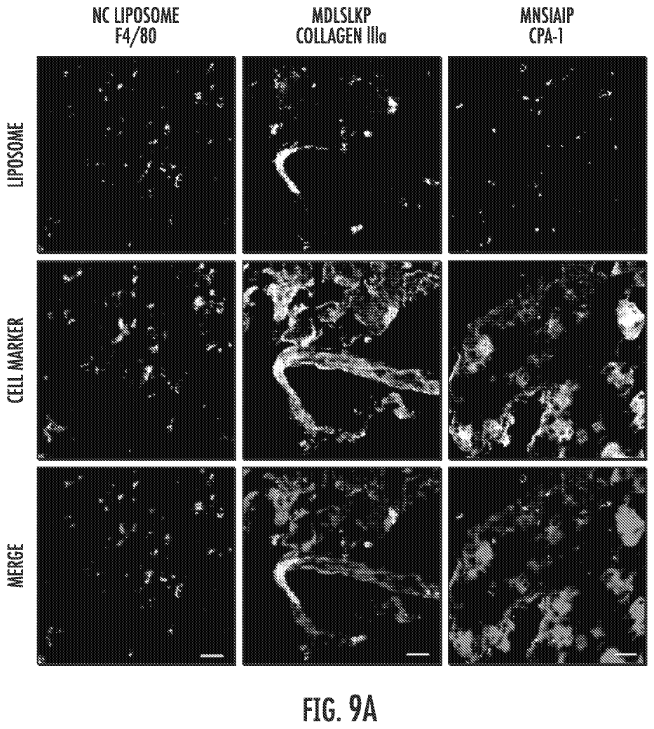

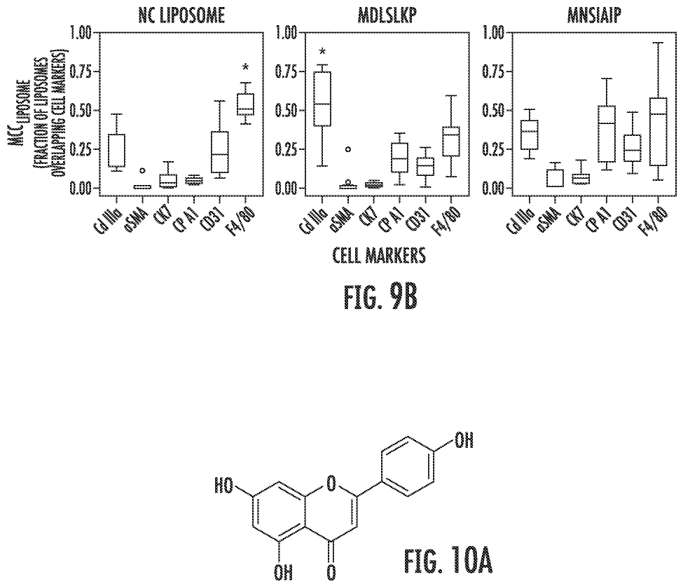

The cellular selectivity of the targeting liposomes in the inflamed pancreas at 48 hours post injection was systematically validated via colocalization analyses on IF-stained tissue sections stained for 6 cell markers (FIG. 9A). As expected, NP liposomes were taken up and colocalized with macrophages (MCC=0.534±0.178) as liposomes were readily phagocytosed by macrophages, which are abundant in the inflamed pancreas (Vonarbourg et al., 2006, Zahednezhad et al., 2019) The addition of peptide ligands shifted the cellular targets of the MDLSLKP (SEQ ID NO: 19) liposome to collagen IIIa⁺ expressing cells (MCC=0.493±0.142), demonstrating the preservation of similar ECM selectivity as it was observed by the phage (FIG. 9B). The MNSIAIP (SEQ ID NO: 20) liposome, however, did not show statistically significant selectivity towards CPA-1+cells, suggesting that this peptide lost its selectivity once conjugated to a liposome. The loss of cell selectivity could explain the result of the in vivo studies where increased pancreas accumulation was observed in the phage form but not in the liposomal form. Combining the pharmacokinetics and cell colocalization results, it was concluded that MDLSLKP (SEQ ID NO: 19) peptide improved liposome targeting to collagen IIIa⁺ expressing cells by 1.5-3 fold as compared with other cell types, resulting in increased pancreas accumulation when compared with non-targeted liposomes. The targeting ligands identified shift liposomes away from macrophage uptake and towards target cells as supported by the Tukey-Kramer test of MCCs of all cell types in the NP and MDLSLKP (SEQ ID NO: 19) liposomes (Table 10).

### EXAMPLE 5

### Targeted Delivery of Apigenin Enhances Anti-fibrotic Effects in CP Mice

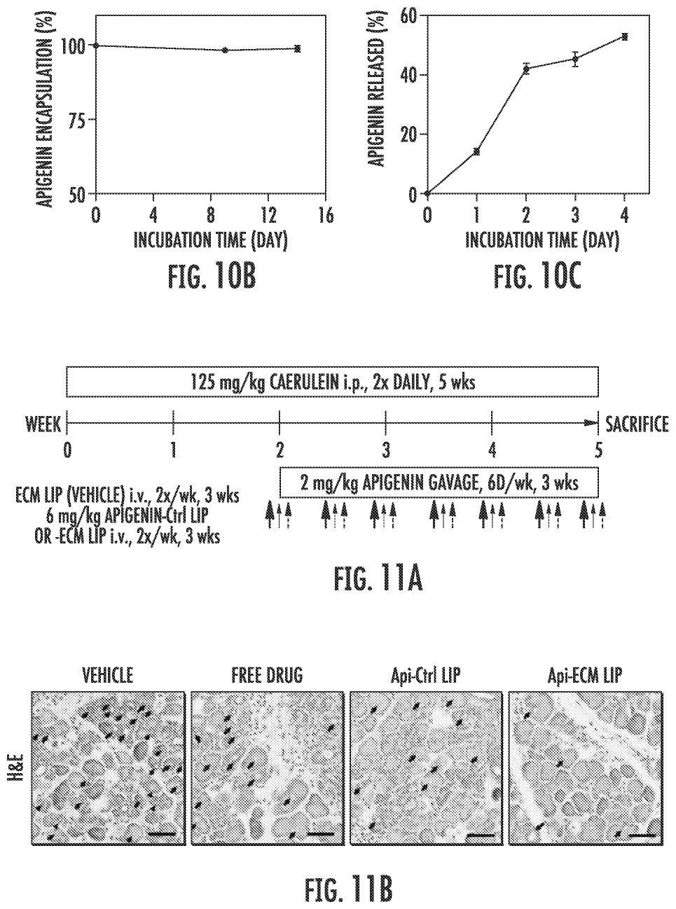

Apigenin is a small molecule natural compound, that has been demonstrated preclinically to have antifibrotic and anti-inflammatory properties in CP (Mrazek et al., 2015; Mrazek et al., 2019). Apigenin, however, suffers from low aqueous solidity, metabolic instability, and off-target effects that there are no approved clinical applications of apigenin available (Srivastava & Gupta, 2009; Singh et al., 2012; Zhang et al., 2012). Therefore, apigenin was used as a proof of concept and directly loaded it into the MDLSLKP (SEQ ID NO: 19) conjugated liposomes (ECM liposome). Physiochemical properties of apigenin are summarized in FIGS. 10B and Table 11. Free apigenin was removed from liposomes by size exclusion chromatography. The drug loading was determined using UV spectrometer with a 40-50% encapsulation rate in both NP and ECM liposomes. The final drug-to-lipid ratio was estimated at an average of 80-100 μg apigenin per mg of lipid, and each liposome contains about 28000 to about 34000 drug per particle (Table 12). Both apigenin-loaded NP and ECM liposomes had a diameter of 90-100 nm measured by NanoSight. The shelf-life of the drug-encapsulated liposomes was also determined and showed >95% of the encapsulated apigenin remained in liposomes in the storage condition (PBS, 4° C.) over 14 days (FIG. 10B).

Hydrophobic molecules are usually encapsulated in the lipid layer of liposomes and can burst release out from the particles in vivo (Gubernator, 2011). To test whether apigenin burst released in the described system, an in vitro release study was performed in 50% FBS/PBS at 37° C. Burst release of apigenin was not observed in the first couple hours upon placing liposomes in 50% FBS. Instead, 14.02% of the encapsulated apigenin molecules were released by day 1 and 52.69% by day 4 (FIG. 10C).

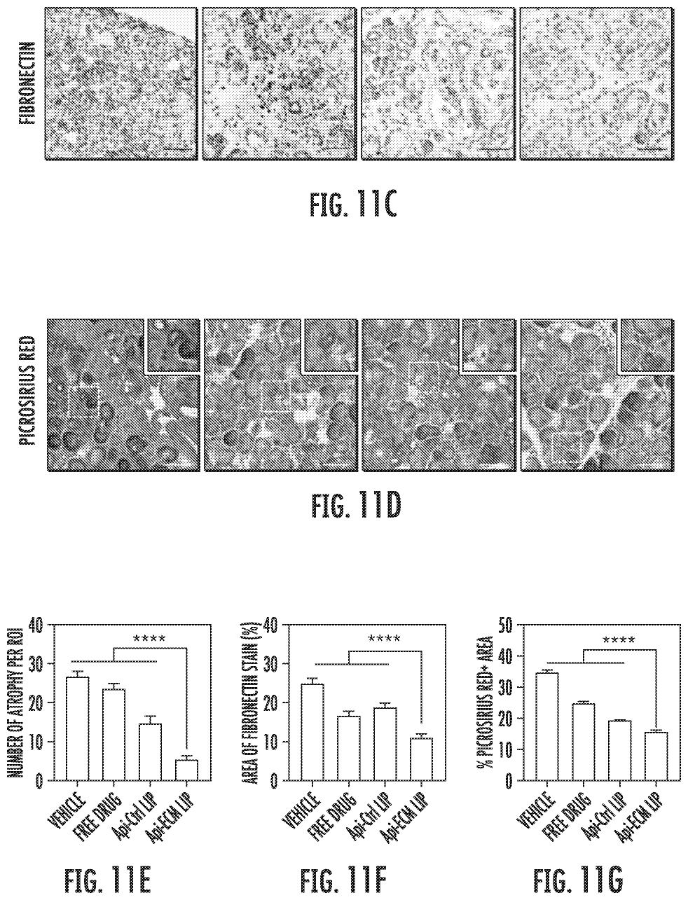

To test the antifibrotic efficacy of targeted liposomes, we evaluated the pharmacodynamics of apigenin in the free drug form, encapsulated in non-targeted liposomes and in ECM liposomes (FIG. 11A). ECM liposomes without drug loading were included in the study as vehicle control. C57BL/6J mice were injected with caerulein 14 days before treatment started to establish inflammation in the pancreas. Apigenin, in the free drug or liposomal form, was given in the remaining 3-week course along with caerulein. As expected, increased acini atrophy and cellular heterogeneity in size and shape, in addition to increased interstitial space, fibrosis, and collagen deposition (34.83% area) were observed in the control group (Mrazek et al., 2015; FIGS. 11B-11D). Compared to free drug and NP liposomes, targeted delivery of apigenin resulted in enhanced preservation of acini units with a respective 4-fold and 2-fold reduction of acinar atrophy counts observed in the pancreas and the decrease of interstitial space between acinus (FIGS. 11B and 11E). Although NP liposomes have substantial macrophage uptake, the enhanced pharmacodynamic activity of apigenin loaded into ECM targeted liposomes demonstrated the importance of cell-specific targeting to drug activity. Using Picrosirius red to stain for collagen, we found that collagen deposition was reduced from 24.90% area to 15.63% in the free apigenin versus targeted liposomal form, respectively (p<0.0001; FIGS. 11D and 11G). Apigenin loaded in NP liposomes reduced collagen to 19.18% area of the inflamed tissue, suggesting that liposomal formulation of apigenin alone resulted in better therapeutic efficacy than free drug but was not as effective as targeted delivery to the ECM. Targeted liposomes resulted in 33.1% better reduction in fibronectin expression when compared to free drug alone and 41.0% reduction compared to NP liposomes (p<0.05. FIGS. 11C and 11F).

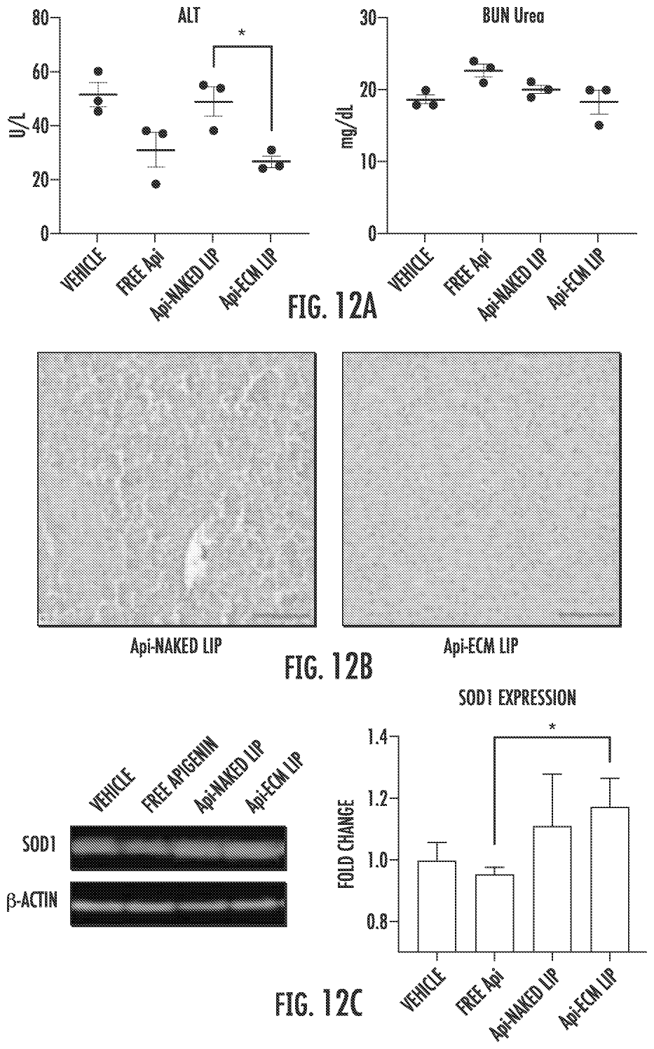

In addition to therapeutic efficacy, hepatotoxicity induced by apigenin (Singh et al., 2012) in free drug and liposomal form was also evaluated and a 1.2-fold increase of SOD1 expression in targeted liposomes was shown, indicating reduction of oxidative stress in the liver (FIG. 12C), matching the reduced liver accumulation found via liposome biodistribution. Liposome-based anti-fibrotic therapies have been evaluated in many pre-clinical studies (Sung et al., 2007; Jose et al., 2016; Khaja et al., 2016; Zhang et al., 2018). Lacking a selective and specific targeting ligand, however, has limited the clinical implementation. The 7-mer peptides identified from the inflamed pancreas can be applied to other cell type-specific therapeutic molecules to potentially improve tissue remodeling and reduce fibrosis and inflammation in CP.

### Discussion of the Examples

Chronic pancreatitis is a complex inflammatory pancreatic disease that remains incurable (Whitcomb, 2016; Whitcomb et al., 2016, Whitcomb et al., 2018; Lowe et al., 2019). Current treatments for CP are limited to palliative care and pain alleviation, and these approaches fail at the advanced stage when invasive surgical procedures such as endoscopie interventions, bypass, and total pancreatectomy are the only available options (D'Haese et al., 2016). The pancreatic community has recently reached a consensus that precision medicine can provide a more sophisticated approach for complex disorders like CP to assist the development of target-specific interventions (Lowe et al., 2019). Despite omics-based technology being widely used to profile disease-specific biomarkers and therapeutic targets in many diseases (Matthews et al., 2016; Hasin et al., 2017), transcriptomics reveals little about CP-specific pathways due to the universal genetic backgrounds shared between pancreatitis, pancreatic cancer, and the benign pancreas (Li et al., 2018). Looking only in epithelial cells, differential expression in 34 proteins in malignant and pancreatitis pancreas compared to the benign tissue has been shown, but these failed to distinguish pancreatitis from pancreatic cancer (Sanh et al., 2018). Considering the heterogeneity of cellular components involved in disease progression of CP, there is a definite need to provide an unbiased, comprehensive evaluation of pancreatitis-associated proteomes.

As described herein, a computational-guided in vivo phage display approach was employed to profile 7-mer peptide ligands specific for cellular components in the caerulein-induced CP mouse pancreas. In contrast to indirect proteomic techniques, phage display allows probing of proteins in their native context during biopanning, thus increasing clinical relevance of the identified targeting agents (Onogi et al., 2020). Additionally, in vivo screening ensures identifying targeting agents with high selectivity as deselections or subtractions for all other tissues are carried out while enrichment occurs in the target tissue (Dasa et al., 2015). By comparing the in vivo screens against CP, benign pancreas, and pancreatic cancer as described herein, it was possible to ensure peptide selectivity to the diseased pancreas by not choosing peptides in any condition but CP. Phage display combined with Illumina NGS overcame conventional biopanning limitations, including high false-positive rates and lack of a robust analytical target selection method (Juds et al., 2020). Using the PHASTpep-guided approach described herein, peptides specific for pancreatic cancer-associated fibroblasts have been successfully identified in vitro (Brinton et al., 2016). As disclosed herein, the application has been expanded in analyzing in vivo phage screens, and the identified phage clones were assessed with live animal imaging modalities and fluorescent microscopy to show peptide specificity towards potential cellular target of interest in CP, including activated PSC, acinar cells, macrophages, and extracellular matrix.

To explore the potential of the CP targeting peptides in cell type-specific drug delivery, peptides were conjugated to pegylated DOPC liposomes and the pharmacokinetics were characterized. Of the two peptides that were conjugated, liposomes displaying ECM and acini targeting peptides shifted the time to reach peak liposomal concentration in the CP pancreas by 24 hours, but did not enhance the overall particle accumulations compared to the NP liposomes. A hallmark of CP is the presence of large numbers of phagocytic cells and indeed, NP liposomes were taken up by the macrophage cell population. The addition of targeting ligands, however, shifted the cellular targets of the liposomes. MDLSLKP (SEQ ID NO: 19) conjugated liposomes demonstrated specificity to collagen IIIa⁺ cells, which is consistent with its phage clone. A shift in organ accumulation from the clearance organs to the inflamed pancreas was also achieved when injecting liposomes displaying MDLSLKP peptides (SEQ ID NO: 19). Disappointingly, cellular selectivity of MNSIAIP (SEQ ID NO: 20) to CPA-1-positive cells was not observed after conjugation to liposomes, suggesting that the current orientation of MNSIAIP (SEQ ID NO: 20) displayed on the liposome may have hindered the binding of the peptide to its target. The lack of selectivity could explain the no statistically significant difference observed in particle accumulation of the MNSIAIP (SEQ ID NO: 20) liposome compared to the NP liposome. This finding emphasizes the importance of characterizing targeted liposome's cellular targets. The ability to specifically target multiple different cell types in CP can open a new avenue for therapeutic strategies that address the crosstalk between ECM components and aPSCs, which initiates multiple cascades of events in fibrogenesis and inflammation in CP (Xiao et al., 2015; Mrazek et al., 2019). Indeed, targeting apigenin to the ECM demonstrated was disclosed to enhance pharmacodynamic effects beyond that of targeting macrophages alone with NP liposomes.

Apigenin is a small molecule drug that has been shown to have antifibrotic, anti-inflammatory, and proapoptotic effects in cancer and chronic inflammatory diseases in vitro and in vivo (Shukla & Gupta, 2010, Mrazek et al., 2015; Chen & Zhao, 2016; Zhang et al., 2017; Mrazek et al., 2019). The clinical use of apigenin, however, is limited by low aqueous solubility, high metabolic instability, and potential hepatotoxicity at acute use (Srivastava & Gupta, 2009; Singh et al., 2012; Zhang et al., 2012). As a proof of concept, we encapsulated apigenin in liposomes and evaluated the antifibrotic effects through cellular component-specific delivery to the ECM in CP pancreas. After 3 consecutive weeks of treatments, mice receiving drug-encapsulated, ECM-targeted liposomes showed the best tissue remodeling effects, including acini unit preservation and stroma reduction, compared to free drug and non-targeted liposomes. In addition, targeted delivery reduced off-target effects (Wu et al., 2015) as mice receiving apigenin encapsulated in targeted liposomes showed reduced ALT and liver SOD1 expression and preserved liver histology compared to the non-targeted delivery (FIGS. 12A and 12B). The presently disclosed results demonstrated that cell type-specific targeting of small molecule drugs, can improve pharmacodynamics and alter the anatomical endpoint (fibrosis) in the inflamed pancreas.

The improvement in increasing the ratio of on-target to off-target effects could address the side effects induced by antifibrotic drugs that act on canonical extracellular factors, including growth factors, cytokines, and MMPs. For example, TGF-β inhibitors are amongst the majority of approved or investigational anti-fibrosis drug families and have demonstrated efficacy in reducing cardiac, liver, and kidney fibrosis (Li et al., 2017). However, galnisertib, a TGF-β R1 kinase inhibitor, caused cardiac toxicity, bone development abnormality and induced irregular inflammatory responses in skin and gut at long-term use, which ended with termination on Phase II clinical trial (NCT0113801; Herbertz et al., 2015). The ability to selectively target multiple different cell types in CP can open a new avenue for therapeutic strategies that address the crosstalk between ECM components and aPSCs, which initiates multiple cascades of events in fibrogenesis and inflammation in CP (Xiao et al., 2015; Mrazek et al., 2019). Indeed, we demonstrated that targeting apigenin to the ECM demonstrated enhanced pharmacodynamic effects beyond that of targeting macrophages alone with NP liposomes, underscoring the importance of targeting.

In conclusion, disclosed herein is a high-throughput approach to guide ligand selection for cellular targets in a diseased mouse model. The present findings (1) revealed five heptapeptides specific to CP; (2) demonstrated that the conjugation of CP-specific peptides to pegylated DOPC liposomes was capable of increasing particle accumulation in the inflamed pancreas; and (3) showed that targeted delivery of apigenin in a mouse model of CP enhanced tissue remodeling, attenuated pancreatic fibrosis, and reduced liver toxicity. It is believed that the presently disclosed subject matter is the first to identify a non-serum-based molecular ligand specific for CP. The ligand identification process was robust and required no prior knowledge of the target, offering the potential to be easily applied to other disease models. The presently disclosed subject matter also provided opportunities for future applications of nanomedicine for the targeting of chronic inflammatory diseases.

### REFERENCES

All references listed in the instant disclosure and/or listed herein below, including but not limited to all patents, patent applications and publications thereof, scientific journal articles, and database entries (including but not limited to UniProt, EMBL, and GENBANK® biosequence database entries and including all annotations available therein) are incorporated herein by reference in their entireties to the extent that they supplement, explain, provide a background for, and/or teach methodology, techniques, and/or compositions employed herein. The discussion of the references is intended merely to summarize the assertions made by their authors. No admission is made that any reference (or a portion of any reference) is relevant prior art. Applicants reserve the right to challenge the accuracy and pertinence of any cited reference.
Abramoff et al. (2004) Image Processing with ImageJ. Biophotonics International 11(7):36-42.
Ahmed et al. (2021) Rationalizing the therapeutic potential of apigenin against cancer. Life Sci 267:118814.
Alavi & Hamidi (2019) Passive and active targeting in cancer therapy by liposomes and lipid nanoparticles. Drug Metab Pers Ther 34(1). doi: 10.1515/dmpt-2018-0032
Andreatta et al. (2017) GibbsCluster: unsupervised clustering and alignment of peptide sequences. Nucleic Acids Res 45: W458-W463.
Bang et al. (2014) Mortality, cancer, and comorbidities associated with chronic pancreatitis: a Danish nationwide matched-cohort study. Gastroenterology 146:989-994.
Bankhead et al. (2017) QuPath: Open source software for digital pathology image analysis. Sci Rep 7:16878.
Bausch et al. (2011) Plectin-1 as a novel biomarker for pancreatic cancer. Clin. Cancer Res. 17:302-309
Berkow et al. (1997) The Merck Manual of Medical Information, Home ed., Merck Research Laboratories, Whitehouse Station, New Jersey, United States of America.
Beyer et al. (2020) Chronic pancreatitis. Lancet Lond Engl 396(10249):499-512.
Bornmann et al. (2008) A new liposomal formulation of Gemcitabine is active in an orthotopic mouse model of pancreatic cancer accessible to bioluminescence imaging. Cancer Chemother Pharmacol 61:395-405.
Bouwense et al. (2019) Surgery in Chronic Pancreatitis: Indication, Timing and Procedures. Visc Med 35(2): 110-118.
Brinton et al. (2016) PHASTpep: Analysis Software for Discovery of Cell-Selective Peptides via Phage Display and Next-Generation Sequencing. PLOS ONE 11:e0155244.
Chen & Zhao (2016) Apigenin protects against bleomycin-induced lung fibrosis in rats. Exp Ther Med 11:230-234.
Chen et al. (2014) Design, synthesis, and characterization of novel apigenin analogues that suppress pancreatic stellate cell proliferation in vitro and associated pancreatic fibrosis in vivo. Bioorg Med Chem 22:3393-3404
Chou & Fasman (1974) Prediction of protein conformation. Biochemistry 13:222-245.
Chou & Fasman (1978) Empirical predictions of protein conformation. Ann Rev Biochem 47:251-276.
Chou & Fasman (1979) Prediction of beta-turns. Biophys J 26:367-384.
D'Haese et al. (2016) J. Current Surgical Treatment Options in Chronic Pancreatitis. Pancreapedia: Exocrine Pancreas Knowledge Base, DOI: 10.3998/panc.2016.26.
Dasa et al. (2015) Development of target-specific liposomes for delivering small molecule drugs after reperfused myocardial infarction. J Control Release 220:556-567.
Deutscher et al. (1990) Guide to Protein Purification, Harcourt Brace Jovanovich, San Diego, California, United States of America.
Duch et al. (1998) Volatile anesthetics significantly suppress central and peripheral mammalian sodium channels. Toxicol Lett 100-101:255-263.
Ebadi (1998) CRC Desk Reference of Clinical Pharmacology. CRC Press, Boca Raton, Florida, United States of America.
Etemad & Whitcomb (2001) Chronic pancreatitis: diagnosis, classification, and new genetic developments. Gastroenterology 120:682-707.
Gabizon et al. (2016) New insights and evolving role of pegylated liposomal doxorubicin in cancer therapy. Drug Resist Updat Rev Comment Antimicrob Anticancer Chemother 29:90-106.
Gardner et al. (2020) ACG Clinical Guideline: Chronic Pancreatitis. Off J Am Coll Gastroenterol ACG 115(3):322-339.
Gerbino (2005) Remington: The Science and Practice of Pharmacy. 21st Edition. Lippincott Williams & Wilkins, Philadelphia, Pennsylvania, United States of America.
Goodman et al. (1996) Goodman & Gilman's the Pharmacological Basis of Therapeutics. 9th ed, McGraw-Hill Health Professions Division, New York, New York, United States of America.
Gubernator (2011) Active methods of drug loading into liposomes: recent strategies for stable drug entrapment and increased in vivo activity. Expert Opin Drug Deliv 8:565-580.
Guo et al. (2013) Identification and Characterization of a Cellulose Binding Heptapeptide Revealed by Phage Display. Biomacromolecules 14(6): 1795-1805.
Gutknecht et al. (2017) Identification of the S100 fused-type protein hornerin as a regulator of tumor vascularity. Nat Commun 8:552.
Halbrook et al. (2017) Mitogen-activated Protein Kinase Kinase Activity Maintains Acinar-to-Ductal Metaplasia and Is Required for Organ Regeneration in Pancreatitis. Cell. Mol. Gastroenterol. Hepatol 3:99-118.
Hall et al. (2014) The socio-economic impact of chronic pancreatitis: a systematic review. J Eval Clin Pract 20:203-207.
Han et al. (2018) Quality of life comparison between smokers and non-smokers with chronic pancreatitis. Pancreatol Off J Int Assoc Pancreatol IAP A1 18(3):269-274.
Hasin et al. (2017) Multi-omics approaches to disease. Genome Biol 18:83.
Herbertz et al. (2015) Clinical development of galunisertib (LY2157299 monohydrate), a small molecule inhibitor of transforming growth factor-beta signaling pathway. Drug Des Devel Ther 9:4479-4499.
Hobohm et al. (1992) Selection of representative protein data sets. Protein Science 1(3):409-417.
http://parts.igem.org/Part:BBa K2043011:Design.
https://metacpan.org/pod/String::Cluster::Hobohm.
Hu et al. (2020) Macrophages in pancreatitis: Mechanisms and therapeutic potential. Biomed Pharmacother Biomedecine Pharmacother 131:110693.
Immordino et al. (2006) Stealth liposomes: review of the basic science, rationale, and clinical applications, existing and potential. Int J Nanomedicine 1(3):297-315.
Jose et al. (2016) Liposomal hydrogel formulation for transdermal delivery of pirfenidone. J Liposome Res 26:139-147.
Juds et al. (2020) Combining Phage Display and Next-Generation Sequencing for Materials Sciences: A Case Study on Probing Polypropylene Surfaces. J Am Chem Soc 142:10624-10628.
Katzung (2001) Basic & Clinical Pharmacology, 8th ed., Lange Medical Books/McGraw-Hill Medical Pub. Division, New York, New York, United States of America.
Kawanishi et al. (2010) Exercise training inhibits inflammation in adipose tissue via both suppression of macrophage infiltration and acceleration of phenotypic switching from M1 to M2 macrophages in high-fat-diet-induced obese mice. Exerc Immunol Rev 16:105-118.
Khaja et al. (2016) Targeted Sterically Stabilized Phospholipid siRNA Nanomedicine for Hepatic and Renal Fibrosis. Nanomaterials 6:8.
Kichler & Jang (2020) Chronic Pancreatitis: Epidemiology, Diagnosis, and Management Updates. Drugs 80(12):1155-1168.
Klöppel (1990) Pathology of chronic pancreatitis and pancreatic pain. Acta Chir Scand 156:261-5.
Koning et al. (2002) Interference of macrophages with immunotargeting of liposomes. J Liposome Res 12(1-2): 107-119.
Kroiss et al. (2019) Unbiased Discovery of Dynamic Peptide-ATP Complexes. ChemSystemsChem 1:7-11.
Kyte & Doolittle (1982) A simple method for displaying the hydropathic character of a protein. J Mol Biol 157:105-132.
Li et al. (2017) Drugs and Targets in Fibrosis. Front Pharmacol 8:855.
Li et al. (2018) The Key Genes of Chronic Pancreatitis which Bridge Chronic Pancreatitis and Pancreatic Cancer Can be Therapeutic Targets. Pathol Oncol Res 24:215-222.
Lowe et al. (2019) Precision Medicine in Pancreatic Disease-Knowledge Gaps and Research Opportunities: Summary of a National Institute of Diabetes and Digestive and Kidney Diseases Workshop. Pancreas 48:1250-1258.
Machicado et al. (2019) Period prevalence of chronic pancreatitis diagnosis from 2001-2013 in the commercially insured population of the United States. Pancreatology 19(6):813-818.
Machicado et al. (2017) Quality of Life in Chronic Pancreatitis is Determined by Constant Pain, Disability/Unemployment, Current Smoking, and Associated Co-Morbidities. Am J Gastroenterol 112:633-642.
Manders et al. (1993) Measurement of co-localization of objects in dual-colour confocal images. J Microsc 169:375-382.
Matthews et al. (2016) “Omics”-Informed Drug and Biomarker Discovery: Opportunities, Challenges and Future Perspectives. Proteomes 4:28.
Morgan et al. (2012) Pain Control and Quality of Life After Pancreatectomy with Islet Autotransplantation for Chronic Pancreatitis. J Gastrointest Surg 16:129-134. Mrazek et al. (2015) Apigenin inhibits pancreatic stellate cell activity in pancreatitis. J Surg Res 196:8-16.
Mrazek et al. (2019) Apigenin Decreases Acinar Cell Damage in Pancreatitis. Pancreas 48:711-718.
Onogi et al. (2020) Identification and characterization of adipose surface epitopes. Biochem J 477:2509-2541.
PCT International Publication Nos. WO 93/25521
Remington et al. (1975) Remington's Pharmaceutical Sciences, 15th ed. Mack Pub. Co., Easton, Pennsylvania, United States of America.
Ryvkin et al. (2018) Phage display peptide libraries: deviations from randomness and correctives. Nucleic Acids Res 46:e52.
Sanh et al. (2018) Proteomics Profiling of Pancreatic Cancer and Pancreatitis for Biomarkers Discovery. J Cell Sci Ther 9:287.
Schneider et al. (2012) NIH Image to ImageJ: 25 years of image analysis. Nature Methods 9:671-675, 2012.
Shukla & Gupta (2010) Apigenin: A Promising Molecule for Cancer Prevention. Pharm Res 27:962-978.
Singh et al. (2012) Acute Exposure of Apigenin Induces Hepatotoxicity in Swiss Mice. PLOS ONE 7:e31964.
Speight & Holford (eds.) (1997) Avery's Drug Treatment: A Guide to the Properties, Choice, Therapeutic Use and Economic Value of Drugs in Disease Management, 4th ed. Adis International, Philadelphia, Pennsylvania, United States of America.
Srivastava & Gupta (2009) Extraction, Characterization, Stability and Biological Activity of Flavonoids Isolated from Chamomile Flowers. Mol Cell Pharmacol 1:138.
Stout et al. (2005) Macrophages sequentially change their functional phenotype in response to changes in microenvironmental influences. J Immunol 175:342-349.
String::Cluster::Hobohm—Cluster strings using the Hobohm algorithm—metacpan.org. https://metacpan.org/pod/String::Cluster::Hobohm.
Sung et al. (2007) Reduction of renal fibrosis as a result of liposome encapsulated clodronate induced macrophage depletion after unilateral ureteral obstruction in rats. Nephron Exp Nephrol 105:el-9.
Ting et al. (2016) Direct Costs of Acute Recurrent and Chronic Pancreatitis in Children in the INSPPIRE Registry. J Pediatr Gastroenterol Nutr 62:443-449.
U.S. Patent Application Publication No. 2006/0188558.

### U.S. Patent Nos.

U.S. Pat. Nos. 3,598,122; 4,554,101; 5,016,652; 5,326,902; 5,234,933; 5,935,975; 6,106,856; 6, 162,459, 6,180,082; 6,495,605; 6,582,724.
Vonarbourg et al. (2006) Evaluation of pegylated lipid nanocapsules versus complement system activation and macrophage uptake. J Biomed Mater Res A 78A:620-628.
Whitcomb (2016) Peering Into the “Black Box” of the Complex Chronic Pancreatitis Syndrome. Pancreas 45:1361-1364.
Whitcomb et al. (2016) Chronic pancreatitis: An international draft consensus proposal for a new mechanistic definition. Pancreatol. Off. J. Int. Assoc. Pancreatol 16:218-224.
Whitcomb et al. (2018) International consensus statements on early chronic Pancreatitis. Recommendations from the working group for the international consensus guidelines for chronic pancreatitis in collaboration with The International Association of Pancreatology, American Pancreatic Association, Japan Pancreas Society, PancreasFest Working Group and European Pancreatic Club. Pancreatol 18:516-527.
Wu et al. (2015) The Targeted-liposome Delivery System of Antitumor Drugs. Curr Drug Metab 16:894-910.
Wynn & Barron (2010) Macrophages: Master Regulators of Inflammation and Fibrosis. Semin Liver Dis 30:245-257.
Xiao et al. (2015) Retinoic Acid Ameliorates Pancreatic Fibrosis and Inhibits the Activation of Pancreatic Stellate Cells in Mice with Experimental Chronic Pancreatitis via Suppressing the Wnt/B-Catenin Signaling Pathway. PLOS ONE 10:e0141462.
Yadav & Lowenfels (2013) The epidemiology of pancreatitis and pancreatic cancer. Gastroenterology 144:1252-1261.
Yang et al. (2020) Macrophage polarization in atherosclerosis. Clin Chim Acta Int J Clin Chem 501:142-146.
Zahednezhad et al. (2019) Liposome and immune system interplay: Challenges and potentials. J Control Release 305:194-209.
Zhang et al. (2012) Biopharmaceutics classification and intestinal absorption study of apigenin. Int J Pharm 436:311-317.
Zhang et al. (2017) The potential application of strategic released apigenin from polymeric carrier in pulmonary fibrosis. Exp Lung Res 43:359-369.
Zhang et al. (2018) Vitamin A-coupled liposomes carrying TLR4-silencing shRNA induce apoptosis of pancreatic stellate cells and resolution of pancreatic fibrosis. J Mol Med 96:445-458.

While the presently disclosed subject matter has been disclosed with reference to specific embodiments, it is apparent that other embodiments and variations of the presently disclosed subject matter may be devised by others skilled in the art without departing from the true spirit and scope of the presently disclosed subject matter.

TABLE 3
Fluorescent Labeling of Candidate Phage Clones

| | | Phage injected (pfu) |
|---|---|---|
| | # Dye Per Phage | equil. to 1 nmol dye |
| GROUP | (CP; Healthy) | (CP; Healthy) |
| 1 | 182.44 ± 29.25 | 3.08 × 10¹¹ ± 1.17 × 10¹⁰ |
| | 191.36 ± 37.08 | 2.93 × 10¹¹ ± 5.44 × 10¹⁰ |
| 2 | 233.43 ± 19.23 | 4.32 × 10¹¹ ± 1.42 × 10¹¹ |
| | 215.99 ± 20.32 | 4.82 × 10¹¹ ± 1.24 × 10¹¹ |
| 3 | 234.32 ± 31.21 | 3.14 × 10¹¹ ± 7.68 × 10¹⁰ |
| | 212.04 ± 35.68 | 4.02 × 10¹¹ ± 5.10 × 10¹⁰ |
| 4 | 240.93 ± 37.01 | 3.36 × 10¹¹ ± 8.78 × 10¹⁰ |
| | 203.53 ± 35.50 | 4.35 × 10¹¹ ± 6.76 × 10¹⁰ |
| WT | 321.09 ± 45.03 | 5.18 × 10¹¹ ± 6.69 × 10¹⁰ |
| | 376.06 ± 20.37 | 3.70 × 10¹¹ ± 2.13 × 10¹⁰ |

TABLE 4
Phage Clone Individual Validation Raw Data and Calculation

| | | | | Targeting Phage-VT680 | | | | Targeting Phage-VT680 | | | | |
|---|---|---|---|---|---|---|---|---|---|---|---|---|
| | | | | M13Ke Phage-VT750 | | | | M13Ke Phage-VT750 | | | | Radiant |
| | | | | | | | Normal- | | | | Normal- | effi- |
| | | | | No. of | | | ized | No. of | | | ized | ciency |
| | | Organ | | Phage | No. of | IVIS | radiant | Phage | No. of | IVIS | radiant | Target- |
| | Mouse | | Weight | In- | dye/ | read- | effi- | In- | dye/ | read- | effi- | ing: |
| Phage | Model | Organ | (mg) | jected | phage | outs# | ciency* | jected | phage | outs# | ciency* | M13Ke |
| HPYSPLR | CP | Liver | 898.12 | 4.0E+11 | 144.97 | 8.81E+09 | 1.69E−04 | 4.0E+11 | 372.35 | 3.82E+10 | 2.86E−04 | 0.59 |
| (SEQ ID | | Pancreas | 391.21 | 4.0E+11 | 144.97 | 7.09E+09 | 3.13E−04 | 4.0E+11 | 372.35 | 2.84E+09 | 4.87E−05 | 6.42 |
| NO: 11) | | Spleen | 67.22 | 4.0E+11 | 144.97 | 3.75E+09 | 9.61E−04 | 4.0E+11 | 372.35 | 3.10E+09 | 3.10E−04 | 3.10 |
| | Healthy | Liver | 394.32 | 4.0E+11 | 140.20 | 1.48E+10 | 6.71E−04 | 4.0E+11 | 406.94 | 5.42E+10 | 8.44E−04 | 0.80 |
| | | Pancreas | 52.36 | 4.0E+11 | 140.20 | 2.77E+09 | 9.44E−04 | 4.0E+11 | 406.94 | 1.94E+09 | 2.27E−04 | 4.16 |
| | | Spleen | 103.91 | 4.0E+11 | 140.20 | 3.92E+09 | 6.73E−04 | 4.0E+11 | 406.94 | 4.18E+09 | 2.47E−04 | 2.73 |
| KTYVPTT | CP | Liver | 854.8 | 4.0E+11 | 167.47 | 1.11E+10 | 1.93E−04 | 4.0E+11 | 372.35 | 4.02E+10 | 3.16E−04 | 0.61 |
| (SEQ ID | | Pancreas | 218.97 | 4.0E+11 | 167.47 | 8.11E+09 | 5.53E−04 | 4.0E+11 | 372.35 | 1.51E+09 | 4.64E−05 | 11.90 |
| NO: 14) | | Spleen | 85.2 | 4.0E+11 | 167.47 | 3.58E+09 | 6.28E−04 | 4.0E+11 | 372.35 | 2.61E+09 | 2.06E−04 | 3.05 |
| | Healthy | Liver | 1067.16 | 4.0E+11 | 167.47 | 1.18E+10 | 1.65E−04 | 4.08+11 | 381.14 | 5.10E+10 | 3.13E−04 | 0.53 |
| | | Pancreas | 410.1 | 4.0E+11 | 167.47 | 3.65E+09 | 1.33E−04 | 4.0E+11 | 381.14 | 9.51E+08 | 1.52E−05 | 8.74 |
| | | Spleen | 98.27 | 4.0E+11 | 167.47 | 7.17E+09 | 1.09E−03 | 4.0E+11 | 381.14 | 4.76E+09 | 3.18E−04 | 3.43 |
| MDLSLKP | CP | Liver | 780.82 | 4.0E+11 | 146.93 | 8.22E+09 | 1.79E−04 | 4.0E+11 | 372.35 | 2.91E+10 | 2.50E−04 | 0.72 |
| (SEQ ID | | Pancreas | 243.54 | 4.0E+11 | 146.93 | 5.85E+09 | 4.09E−04 | 4.0E+11 | 372.35 | 1.37E+09 | 3.77E−05 | 10.85 |
| NO: 19) | | Spleen | 80.31 | 4.0E+11 | 146.93 | 5.25E+09 | 1.11E−03 | 4.0E+11 | 372.35 | 4.92E+09 | 4.11E−04 | 2.70 |
| | Healthy | Liver | 1012.42 | 4.0E+11 | 146.93 | 8.59E+09 | 1.44E−04 | 4.0E+11 | 372.35 | 4.16E+10 | 2.76E−04 | 0.52 |
| | | Pancreas | 264.06 | 4.0E+11 | 146.93 | 4.36E+09 | 2.81E−04 | 4.0E+11 | 372.35 | 1.91E+09 | 4.85E−05 | 5.79 |
| | | Spleen | 91.74 | 4.0E+11 | 146.93 | 3.35E+09 | 6.22E−04 | 4.0E+11 | 372.35 | 3.64E+09 | 2.66E−04 | 2.34 |
| SLPLGPM | CP | Liver | 1006.12 | 4.0E+11 | 257.35 | 1.81E+10 | 1.75E−04 | 4.0E+11 | 372.35 | 4.12E+10 | 2.75E−04 | 0.64 |
| (SEQ ID | | Pancreas | 322.24 | 4.0E+11 | 257.35 | 7.93E+09 | 2.39E−04 | 4.0E+11 | 372.35 | 1.79E+09 | 3.72E−05 | 6.42 |
| NO: 27) | | Spleen | 90.42 | 4.0E+11 | 257.35 | 9.12E+09 | 9.79E−04 | 4.0E+11 | 372.35 | 3.78E+09 | 2.81E−04 | 3.49 |
| | Healthy | Liver | 980.82 | 4.0E+11 | 257.35 | 2.24E+10 | 2.21E−04 | 4.0E+11 | 381.14 | 4.79E+10 | 3.20E−04 | 0.69 |
| | | Pancreas | 343.27 | 4.0E+11 | 257.35 | 3.83E+09 | 1.08E−04 | 4.0E+11 | 381.14 | 1.13E+09 | 2.17E−05 | 5.00 |
| | | Spleen | 95.61 | 4.0E+11 | 257.35 | 7.60E+09 | 7.72E−04 | 4.0E+11 | 381.14 | 3.92E+09 | 2.69E−04 | 2.87 |
| HSGLNKQ | CP | Liver | 240.02 | 4.0E+11 | 141.41 | 8.85E+09 | 6.51E−04 | 4.0E+11 | 372.81 | 3.70E+10 | 1.03E−03 | 0.63 |
| (SEQ ID | | Pancreas | 40.94 | 4.0E+11 | 141.41 | 4.99E+09 | 2.15E−03 | 4.0E+11 | 372.81 | 2.17E+09 | 3.55E−04 | 6.07 |
| NO: 12) | | Spleen | 802.42 | 4.0E+11 | 141.41 | 4.69E+09 | 1.03E−04 | 4.0E+11 | 372.81 | 3.62E+09 | 3.02E−05 | 3.42 |
| | Healthy | Liver | 842.48 | 4.0E+11 | 106.78 | 6.03E+09 | 1.68E−04 | 4.0E+11 | 372.35 | 4.18B+10 | 3.33E−04 | 0.50 |
| | | Pancreas | 239.5 | 4.0E+11 | 106.78 | 4.25E+09 | 4.16E−04 | 4.0E+11 | 372.35 | 1.49E+09 | 4.19E−05 | 9.93 |
| | | Spleen | 90.63 | 4.0E+11 | 106.78 | 3.45E+09 | 8.92E−04 | 4.0E+11 | 372.35 | 4.07E+09 | 3.01E−04 | 2.96 |
| MNSIAIP | CP | Liver | 257.09 | 4.0E+11 | 115.32 | 9.36E+09 | 7.89E−04 | 4.0E+11 | 372.81 | 4.96E+10 | 1.29E−03 | 0.61 |
| (SEQ ID | | Pancreas | 45.9 | 4.0E+11 | 115.32 | 6.63E+09 | 3.13E−03 | 4.0E+11 | 372.81 | 2.19E+09 | 3.19E−04 | 9.81 |
| NO: 20) | | Spleen | 887.72 | 4.0E+11 | 115.32 | 2.27E+09 | 5.54E−05 | 4.0E+11 | 372.81 | 2.03E+09 | 1.53E−05 | 3.61 |
| | Healthy | Liver | 758.75 | 4.0E+11 | 151.45 | 8.22E+09 | 1.79E−04 | 4.0E+11 | 372.35 | 3.79E+10 | 3.35E−04 | 0.53 |
| | | Pancreas | 309.56 | 4.0E+11 | 151.45 | 3.98E+09 | 2.12E−04 | 4.0E+11 | 372.35 | 1.94E+09 | 4.20E−05 | 5.04 |
| | | Spleen | 77.89 | 4.0E+11 | 151.45 | 3.93E+09 | 8.34E−04 | 4.0E+11 | 372.35 | 2.22B+09 | 1.91E−04 | 4.36 |
| SANITNL | CP | Liver | 377.24 | 4.0E+11 | 136.80 | 1.31E+10 | 6.35E−04 | 4.0E+11 | 372.81 | 4.82E+10 | 8.57E−04 | 0.74 |
| (SEQ ID | | Pancreas | 35.65 | 4.0E+11 | 136.80 | 3.74E+09 | 1.92E−03 | 4.0E+11 | 372.81 | 2.54E+09 | 4.77E−04 | 4.02 |
| NO: 25) | | Spleen | 867.87 | 4.0E+11 | 136.80 | 5.11E+09 | 1.08E−04 | 4.0E+11 | 372.81 | 3.03E+09 | 2.34E−05 | 4.60 |
| | Healthy | Liver | 829.3 | 4.0E+11 | 87.24 | 6.56E+09 | 2.27E−04 | 4.0E+11 | 372.35 | 4.86E+10 | 3.93E−04 | 0.58 |
| | | Pancreas | 375.21 | 4.0E+11 | 87.24 | 3.27E+09 | 2.50E−04 | 4.0E+11 | 372.35 | 3.27E+09 | 5.85E−05 | 4.27 |
| | | Spleen | 87.12 | 4.0E+11 | 87.24 | 2.61E+09 | 8.59E−04 | 4.0E+11 | 372.35 | 3.68E+09 | 2.83E−04 | 3.03 |
| SLTNSSF | CP | Liver | 961.08 | 2.5E+11 | 182.53 | 1.82E+10 | 4.43E−04 | 2.5E+11 | 381.14 | 3.01E+10 | 3.34E−04 | 1.33 |
| (SEQ ID | | Pancreas | 217.65 | 2.5E+11 | 182.53 | 1.35E+09 | 1.45E−04 | 2.5E+11 | 381.14 | 3.33E+08 | 1.63E−05 | 8.86 |
| NO: 27) | | Spleen | 93.18 | 2.5E+11 | 182.53 | 1.11E+09 | 2.78E−04 | 2.5E+11 | 381.14 | 9.19E+08 | 1.05E−04 | 2.65 |
| | Healthy | Liver | 810.23 | 4.0E+11 | 447.23 | 9.27E+09 | 6.39E−05 | 4.0E+11 | 372.35 | 7.25E+09 | 6.01E−05 | 1.06 |
| | | Pancreas | 314.3 | 4.0E+11 | 447.23 | 7.86E+09 | 1.40E−04 | 4.0E+11 | 372.35 | 4.04E+09 | 8.63E−05 | 1.62 |
| | | Spleen | 108.22 | 4.0E+11 | 447.23 | 7.59E+09 | 3.92E−04 | 4.0E+11 | 372.35 | 2.35E+09 | 1.46E−04 | 2.69 |
| SNSQDLH | CP | Liver | 302.49 | 4.0E+11 | 167.62 | 1.31E+10 | 6.45E−04 | 4.0E+11 | 372.81 | 3.98E+10 | 8.82E−04 | 0.73 |
| (SEQ ID | | Pancreas | 28.81 | 4.0E+11 | 167.62 | 4.05E+09 | 2.10E−03 | 4.0E+11 | 372.81 | 1.45E+09 | 3.37E−04 | 6.22 |
| NO: 28) | | Spleen | 962.65 | 4.0E+11 | 167.62 | 7.33E+09 | 1.13E−04 | 4.0E+11 | 372.81 | 4.98E+09 | 3.47E−05 | 3.27 |
| | Healthy | Liver | 820.63 | 4.0E+11 | 142.64 | 8.55E+09 | 1.82E−04 | 4.0E+11 | 372.35 | 4.35E+10 | 3.56E−04 | 0.51 |
| | | Pancreas | 336.62 | 4.0E+11 | 142.64 | 1.43E+09 | 7.43E−05 | 4.0E+11 | 372.35 | 1.80E+09 | 3.59E−05 | 2.07 |
| | | Spleen | 70.03 | 4.0E+11 | 142.64 | 2.80E+09 | 7.00E−04 | 4.0E+11 | 372.35 | 2.35E+09 | 2.25E−04 | 3.11 |

#total radiant efficiency
*Normalized radiant efficiency = total radiant efficiency divided by number of phage injected × number of dye/phage × tissue weight in grams)

TABLE 5
Individual Clone Validation
CP-to-healthy Ratio

| | CP-to-Healthy Ratio | | |
|---|---|---|---|
| Phage | Liver | Pancreas | Spleen |
| HPYSPLR | 0.744 | 1.544 | 1.138 |
| SEQ ID NO: 11 | | | |
| KTYVPTT | 1.161 | 1.362 | 0.890 |
| SEQ ID NO: 14 | | | |
| MDLSLKP | 1.370 | 1.875 | 1.156 |
| SEQ ID NO: 19 | | | |
| SLPLGPM | 0.921 | 1.286 | 1.215 |
| SEQ ID NO: 26 | | | |
| HSGLNKQ | 1.254 | 0.611 | 1.157 |
| SEQ ID NO: 12 | | | |
| MNSIAIP | 1.144 | 1.945 | 0.829 |
| SEQ ID NO: 20 | | | |
| SANITNL | 1.287 | 0.943 | 1.518 |
| SEQ ID NO: 25 | | | |
| SLTNSSF | 1.248 | 5.469 | 0.985 |
| SEQ ID NO: 27 | | | |
| SNSQDLH | 1.428 | 3.009 | 0.165 |
| SEQ ID NO: 28 | | | |

CP-to-Healthy ratio was calculated as normalized radiant efficiency of CP divided by normalized radiant efficiency of healthy tissue.

TABLE 6
Summary of Phage Clones Showing Specificity to
Cellular Components in the CP Microenvironment

| Cell type | Marker | Phage Clones |
|---|---|---|
| Acinar cells | CPA1⁺ | MNSIAIP (SEQ ID NO: 20) |
| aPSC | α-SMA⁺ | KTYVPTT (SEQ ID NO: 14) |
| ECM | Collagen | MDLSLKP (SEQ ID NO: 19) |
| | IIIa⁺ | |
| Macrophage | F4/80⁺ | SLINSSF (SEQ ID NO: 27) |
| | | SNSQDLH (SEQ ID NO: 28) |

TABLE 7
P-values for Statistical Tests Performed
on Manders' Correlation Coefficients
of Phage Clones Overlapping with Cell Markers

| Phage | | Adjusted | |
|---|---|---|---|
| Clone | MCC Comparison | P-value | Summary |
| MDLSLKP | Col IIIa vs αSMA | <0.0001 | **** |
| (SEQ ID | Col IIIa vs CD31 | <0.0001 | **** |
| NO: 19) | Col IIIa vs CK7 | <0.0001 | **** |
| | Col IIIa vs CPA-1 | <0.0001 | **** |
| | Col IIIa vs F4/80 | <0.0001 | **** |
| SLPLGPM | F4/80 vs αSMA | 0.0023 | ** |
| (SEQ ID | F4/80 vs CD31 | 0.0448 | * |
| NO: 26) | F4/80 vs CK7 | <0.0001 | **** |
| | F4/80 vs Col IIIa | 0.0284 | * |
| | F4/80 vs CPA-1 | 0.4832 | n.s. |
| HPYSPLR | F4/80 vs αSMA | <0.0001 | **** |
| (SEQ ID | F4/80 vs CD31 | 0.8189 | n.s. |
| NO: 11) | F4/80 vs CK7 | <0.0001 | **** |
| | F4/80 vs Col IIIa | 0.082 | n.s. |
| | F4/80 vs CPA-1 | 0.0342 | * |
| KTYVPTT | αSMA vs CD31 | <0.0001 | **** |
| (SEQ ID | αSMA vs CK7 | <0.0001 | **** |
| NO: 16) | αSMA vs Col IIIa | 0.0003 | *** |
| | αSMA vs CPA-1 | 0.0006 | *** |
| | αSMA vs F4/80 | 0.0095 | ** |
| SLTNSSF | F4/80 vs αSMA | <0.0001 | **** |
| (SEQ ID | F4/80 vs CD31 | <0.0001 | **** |
| NO: 27) | F4/80 vs CK7 | <0.0001 | **** |
| | F4/80 vs Col IIIa | <0.0001 | **** |
| | F4/80 vs CPA-1 | <0.0001 | **** |
| MNSIAIP | CPA-1 vs αSMA | <0.0001 | **** |
| (SEQ ID | CPA-1 vs CD31 | <0.0001 | **** |
| NO: 20) | CPA-1 vs CK7 | <0.0001 | **** |
| | CPA-1 vs Col IIIa | <0.0001 | **** |
| | CPA-1 vs F4/80 | 0.0002 | *** |
| SNSQDLH | F4/80 vs αSMA | <0.0001 | **** |
| (SEQ ID | F4/80 vs CD31 | <0.0001 | **** |
| NO: 28) | F4/80 vs CK7 | <0.0001 | **** |
| | F4/80 vs Col IIIa | <0.0001 | **** |
| | F4/80 vs CPA-1 | <0.0001 | **** |

n.s.: not significant

TABLE 8
Heatmap of Mean Manders' Correlation
Coefficient (MCC) Representing the
Fraction of Phage Overlapping Cell Markers

| | MDLSLKP | SLPLGPM | HPYSPLR | KTYVPTT | SLTNSSF | MNSIAIP | SNSQDLH |
|---|---|---|---|---|---|---|---|
| | SEQ ID | SEQ ID | SEQ ID | SEQ ID | SEQ ID | SEQ ID | SEQ ID |
| | NO: 19 | NO: 26 | NO: 11 | NO: 14 | NO: 27 | NO: 20 | NO: 28 |
| αSMA | 0.16 | 0.15 | 0.13 | 0.52 | 0.06 | 0.07 | 0.05 |
| CD31 | 0.20 | 0.25 | 0.35 | 0.20 | 0.13 | 0.24 | 0.12 |
| CK7 | 0.06 | 0.06 | 0.08 | 0.06 | 0.11 | 0.05 | 0.14 |
| Col IIIa | 0.83 | 0.23 | 0.27 | 0.23 | 0.33 | 0.34 | 0.33 |
| CPA-1 | 0.31 | 0.35 | 0.25 | 0.26 | 0.22 | 0.63 | 0.23 |
| F4/80 | 0.46 | 0.49 | 0.41 | 0.31 | 0.80 | 0.37 | 0.80 |

Manders' colocalization analysis was performed using the ImageJ plug-in JACoP.
n = 10-12 images per marker, per clone.
Col IIIa: collagen IIIa.

TABLE 9
Phage Clones Showing Statistically
Significant Selectivity for a
Single Cellular Component in CP

| | Colocalized | Corresponding |
|---|---|---|
| Phage | Markers | Cellular |
| Clone | (p < 0.05) | Components |
| MDLSLKP | Collagen | ECM |
| SEQ ID NO: 19 | IIIa | |
| KTYVPTT | α-SMA | aPSC |
| SEQ ID NO: 14 | | |
| MNSIAIP | CPA-1 | Acinar cells |
| SEQ ID NO: 20 | | |
| SLTNSSF | F4/80 | Macrophage |
| SEQ ID NO: 27 | | |
| SNSQDLH | F4/80 | Macrophage |
| SEQ ID NO: 28 | | |

One-way ANOVA and Tukey-Kramer tests were used to compare MCC of all cell markers for each clone.
The result was considered significant if p ≤ 0.05.

TABLE 10
Characteristic Features of Surface-modified
Liposomes with Peptides Identified
to Target Collagen IIIa⁺ and Acinar Cells

| | | MDLSLKP | MNSIAIP |
|---|---|---|---|
| | | SEQ ID | SEQ ID |
| Liposome | No Peptide | NO: 19 | NO: 20 |
| Size (nm) | 103.3 ± 1.3 | 105.1 ± 3.1 | 110.5 ± 1.7 |
| Concentration | 1.30 × 10¹⁴ | 8.18 × 10¹³ | 1.47 × 10¹⁴ |
| (particles/mL) | | | |
| Number of | — | 436.52 | 405.05 |
| Peptides/ | | | |
| Liposome | | | |
| Number of | 372.20 | 429.30 | 358.21 |
| DiD/Liposome | | | |
| Zeta Potential | -37.2 | -29.9 | -30.7 |
| (mV) | | | |

TABLE 11
P-values for Statistical Tests Performed
on Manders' Correlation Coefficients
of Liposomes Overlapping with Cell Markers

| Phage | | Adjusted | |
|---|---|---|---|
| Clone | MCC Comparison | P-value | Summary |
| MDLSLKP | Col IIIa vs αSMA | <0.0001 | **** |
| (SEQ ID | Col IIIa vs CD31 | <0.0001 | **** |
| NO: 19) | Col IIIa vs CK7 | <0.0001 | **** |
| | Col IIIa vs CPA-1 | <0.0001 | **** |
| | Col IIIa vs F4/80 | 0.0012 | ** |
| MNSIAIP | CPA-1 vs αSMA | 0.0005 | *** |
| (SEQ ID | CPA-1 vs CD31 | 0.3179 | n.s. |
| NO: 20) | CPA-1 vs CK7 | 0.0013 | ** |
| | CPA-1 vs Col IIIa | 0.9882 | n.s. |
| | CPA-1 vs F4/80 | 0.9746 | n.s. |
| NC | F4/80 vs αSMA | <0.0001 | **** |
| Liposome | F4/80 vs CD31 | <0.0001 | **** |
| | F4/80 vs CK7 | <0.0001 | **** |
| | F4/80 vs Col IIIa | <0.0001 | **** |
| | F4/80 vs CPA-1 | <0.0001 | **** |

n.s.: not significant

TABLE 12
Physiochemical Properties of Apigenin Reported on PubChem

| Properties | Apigenin, C₁₅H₁₀O₅ |
|---|---|
| Molecular Weight | 270.24 g/mol |
| Water Solubility | 0.183 g/mL |
| Charge (ph 7.4) | 0 |
| pKa | pKa₁ = 7.12; pKa₂ = 8.10 |
| Log P | 1.7 |

TABLE 13
Properties of Apigenin-loaded Liposomes

| | No Peptide Liposomes | ECM Liposomes |
|---|---|---|
| Size (nm) | 93.1 ± 3.6 | 94.3 ± 1.4 |
| Batch Concentration | 1.09 × 10¹⁴ | 1.52 × 10¹⁴ |
| (particles/mL) | | |
| Drug per Liposome | 33930 | 28501 |

1. A liposomal composition comprising a liposome, wherein a surface of the liposome has been conjugated to a peptide selected from the group consisting of SEQ ID NOs: 3-38, optionally wherein the liposome encapsulates an active agent selected from the group consisting of a therapeutic agent and a detectable agent.

2. The liposomal composition of claim 1, wherein the peptide is selected from the group consisting of KTYVPTT (SEQ ID NO: 14), MDLSLKP (SEQ ID NO: 19), MNSIAIP (SEQ ID NO: 20) , SLTNSSF (SEQ ID NO: 27), and SNSQDLH (SEQ ID NO: 28).

3. The liposome composition of claim 2, wherein the peptide is MDLSLKP (SEQ ID NO: 19) and the liposome is targeted to a cell expressing collagen IIIa.

4. The liposome composition of claim 1, wherein the therapeutic agent is an antifibrotic agent, optionally apigenin.

5. The liposome composition of claim 1, further comprising a pharmaceutically acceptable carrier, diluent, and/or excipient, optionally, wherein the pharmaceutically acceptable carrier, diluent, and/or excipient is pharmaceutically acceptable for use in a subject.

6. A peptide comprising, consisting essentially of, or consisting of an amino acid sequence as set forth in any one of SEQ ID NOs: 3-38 or a mimetic, analog, or derivative thereof, optionally wherein the amino acid sequence is no more than 10 amino acids, 20 amino acids, 30 amino acids, 40 amino acids, or 50 amino acids.

7. The peptide of claim 6, wherein one or more of the amino acids in the amino acid sequence is a modified amino acid and/or a non-standard amino acid, further optionally wherein the amino acid sequence as set forth in any one of SEQ ID NOs: 3-38 is unmodified.

8. The peptide of claim 6, wherein the peptide is selected from the group consisting of KTYVPTT (SEQ ID NO: 14), MDLSLKP (SEQ ID NO: 19), MNSIAIP (SEQ ID NO: 20) , SLTNSSF (SEQ ID NO: 27), and SNSQDLH (SEQ ID NO: 28).

9. A method for treating or preventing fibrosis, the method comprising administering to a subject in need thereof a therapeutically effective amount of a liposomal composition of claim 1.

10. The method of claim 9, wherein the peptide is MDLSLKP (SEQ ID NO: 19 and the liposome targets a cell expressing collagen IIIa that is present in the subject.

11. The method of claim 9, wherein the cell is present in the pancreas of the subject and the subject is at risk for developing chronic pancreatitis (CP) and/or pancreatic fibrosis.

12. A method for decreasing the incidence of a disease, disorder, or condition associated with chronic pancreatitis (CP), the method comprising administering to a subject in need thereof a therapeutically effective amount of a liposomal composition of claim 1.

13. The method of claim 12, wherein the peptide is MDLSLKP (SEQ ID NO: 19) and the liposome targets a cell expressing collagen IIIa that is present in the subject.

14. The method of claim 11, wherein the disease, disorder, or condition associated with CP is selected from the group consisting of pulmonary disease, diabetes mellitus, and pancreatic cancer.

15. A method for targeting an active agent to a target, the method comprising contacting the target with a liposomal composition of claim 1.

16. The method of claim 15, wherein the target is selected from the group consisting of an acinar cell, an activated pancreatic stellate cell (aPSC), a component of the extracellular matrix (ECM), and a macrophage.

17. The method of claim 15, wherein the liposomal composition comprises a peptide comprising, consisting essentially of, or consisting of an amino acid sequence as set forth in any one of SEQ ID NOs: 14, 19, 20, 27, and 28.

18. The method of claim 15, wherein the active agent is a therapeutic agent, optionally an antifibrotic agent and/or a chemotherapeutic agent, or a detectable agent.

19. A method for decreasing incidence of a side effect associated with apigenin treatment in a subject, the method comprising administering to the subject a liposome, wherein a surface of the liposome has been conjugated to a peptide selected from the group consisting of SEQ ID NOs: 3-38, and further wherein the liposome encapsulates the apigenin.

20. The method of claim 19, wherein the side effect associated with apigenin treatment comprises hepatotoxicity.

21. The method of claim 19, wherein the subject has or is at risk of developing chronic pancreatitis (CP) and/or a disease, disorder, and/or condition associated with CP.

22. A method for targeting an active agent to a collagen III-expressing cell, the method comprising contacting a collagen III-expressing cell with a vehicle comprising the active agent and a peptide comprising, consisting essentially of, or consisting of the amino acid sequence MDLSLKP (SEQ ID NO: 19), wherein the peptide binds to collagen III on or in the cell to thereby target the active agent to the collagen III-expressing cell.

23. The method of claim 22, wherein the collagen III is present in the extracellular matrix of the cell.

24. The method of claim 22, wherein the cell is present in a subject, optionally in the pancreas of the subject.

25. The method of claim 22, wherein the cell is present in the pancreas of the subject.

26. The method of claim 25, wherein the subject has or is at risk of developing chronic pancreatitis (CP) and/or a disease, disorder, and/or condition associated with CP.

27. A method for delivering an active agent to a target, optionally a target in a subject, the method comprising contacting the target with a vehicle comprising the active agent and a peptide comprising, consisting essentially of, or consisting of an amino acid sequence as set forth in any of SEQ ID NOs: 14, 19, 20, 27, and 28, wherein the peptide binds to the target to thereby deliver the active agent to the target.

28. The method of claim 27, wherein the target is selected from the group consisting of:
(i) a collagen III antigen, optionally a collagen III antigen that is present in the extracellular matrix of a cell, and the peptide comprises, consists essentially of, or consists of SEQ ID NO: 19;
(ii) a carboxypeptidase A1 (CPA-1) antigen, optionally a CPA-1 antigen that is present on a pancreatic acinar cell, and the peptide comprises, consists essentially of, or consists of SEQ ID NO: 20;
(iii) an α-smooth muscle actin (α-SMA) antigen, optionally an α-SMA antigen that is present on a cell, and the peptide comprises, consists essentially of, or consists of SEQ ID NO: 14; and
(iv) an F4/80 antigen, optionally an F4/80 antigen that is present on a cell, and the peptide comprises, consists essentially of, or consists of SEQ ID NO: 27 and/or SEQ ID NO: 28.

29. The method of claim 27, wherein the vehicle comprises a plurality of peptides comprising, consisting essentially of, or consisting of at least two amino acid sequence selected from the group consisting of SEQ ID NOs: 14, 19, 20, 27, and 28.

30. The method of claim 27, wherein the target is a cell present in a subject, optionally in the pancreas of the subject.

31. The method of claim 27, wherein the cell is present in the pancreas of the subject.

32. The method of claim 31, wherein the subject has or is at risk of developing chronic pancreatitis (CP) and/or a disease, disorder, and/or condition associated with CP.

33. A method for delivering an active agent to extracellular matrix (ECM), optionally ECM present in a subject, the method comprising contacting the ECM with a vehicle comprising the active agent and a peptide comprising, consisting essentially of, or consisting of an amino acid sequence as set forth in SEQ ID NO: 19, wherein the peptide binds to the ECM to thereby deliver the active agent to the target.

34. The method of claim 33, wherein the ECM is associated with fibrosis in the subject, and the active agent treats or prevents the development and/or progression of the fibrosis in the subject.

35. The method of claim 22, wherein the fibrosis in the subject is associated with chronic pancreatitis (CP) and/or a disease, disorder, and/or condition associated with CP, pancreatic cancer, pulmonary disease, diabetes mellitus, hepatic fibrosis, or any combination thereof.


SEQUENCE LISTING (65 sequences)

SEQ ID NO: 1 - DNA, 21 bp, Artificial Sequence
  Artificially synthesized oligonucleotide sequence
cctttagtggtacctttctat

SEQ ID NO: 2 - DNA, 21 bp, Artificial Sequence
  Artificially synthesized oligonucleotide sequence
gccctcatagttagcgtaacg

SEQ ID NO: 3 - protein, 7 aa, Artificial Sequence
  Artificially synthesized peptide sequence
ADARYKS

SEQ ID NO: 4 - protein, 5 aa, Artificial Sequence
  Artificially synthesized peptide sequence
GGSKC

SEQ ID NO: 5 - protein, 7 aa, Artificial Sequence
  Artificially synthesized peptide sequence
GLKHSPR

SEQ ID NO: 6 - protein, 7 aa, Artificial Sequence
  Artificially synthesized peptide sequence
GSHARND

SEQ ID NO: 7 - protein, 7 aa, Artificial Sequence
  Artificially synthesized peptide sequence
HLDSNKQ

SEQ ID NO: 8 - protein, 7 aa, Artificial Sequence
  Artificially synthesized peptide sequence
HLKANDQ

SEQ ID NO: 9 - protein, 7 aa, Artificial Sequence
  Artificially synthesized peptide sequence
HLTSNYV

SEQ ID NO: 10 - protein, 7 aa, Artificial Sequence
  Artificially synthesized peptide sequence
HNPSKSV

SEQ ID NO: 11 - protein, 7 aa, Artificial Sequence
  Artificially synthesized peptide sequence
HPYSPLR

SEQ ID NO: 12 - protein, 7 aa, Artificial Sequence
  Artificially synthesized peptide sequence
HSGLNKQ

SEQ ID NO: 13 - protein, 7 aa, Artificial Sequence
  Artificially synthesized peptide sequence
IAPSWNV

SEQ ID NO: 14 - protein, 7 aa, Artificial Sequence
  Artificially synthesized peptide sequence
KTYVPTT

SEQ ID NO: 15 - protein, 7 aa, Artificial Sequence
  Artificially synthesized peptide sequence
KSVISDI

SEQ ID NO: 16 - protein, 7 aa, Artificial Sequence
  Artificially synthesized peptide sequence
KTYVPTT

SEQ ID NO: 17 - protein, 7 aa, Artificial Sequence
  Artificially synthesized peptide sequence
LPSTTTA

SEQ ID NO: 18 - protein, 7 aa, Artificial Sequence
  Artificially synthesized peptide sequence
LVWPAPN

SEQ ID NO: 19 - protein, 7 aa, Artificial Sequence
  Artificially synthesized peptide sequence
MDLSLKP

SEQ ID NO: 20 - protein, 7 aa, Artificial Sequence
  Artificially synthesized peptide sequence
MNSIAIP

SEQ ID NO: 21 - protein, 7 aa, Artificial Sequence
  Artificially synthesized peptide sequence
NTAARIP

SEQ ID NO: 22 - protein, 7 aa, Artificial Sequence
  Artificially synthesized peptide sequence
QMHAKYP

SEQ ID NO: 23 - protein, 7 aa, Artificial Sequence
  Artificially synthesized peptide sequence
QMHARGD

SEQ ID NO: 24 - protein, 7 aa, Artificial Sequence
  Artificially synthesized peptide sequence
QMPARDC

SEQ ID NO: 25 - protein, 7 aa, Artificial Sequence
  Artificially synthesized peptide sequence
SANITNL

SEQ ID NO: 26 - protein, 7 aa, Artificial Sequence
  Artificially synthesized peptide sequence
SLPLGPM

SEQ ID NO: 27 - protein, 7 aa, Artificial Sequence
  Artificially synthesized peptide sequence
SLTNSSF

SEQ ID NO: 28 - protein, 7 aa, Artificial Sequence
  Artificially synthesized peptide sequence
SNSQDLH

SEQ ID NO: 29 - protein, 7 aa, Artificial Sequence
  Artificially synthesized peptide sequence
STGTGEH

SEQ ID NO: 30 - protein, 7 aa, Artificial Sequence
  Artificially synthesized peptide sequence
TQSTLHK

SEQ ID NO: 31 - protein, 7 aa, Artificial Sequence
  Artificially synthesized peptide sequence
TYTARGD

SEQ ID NO: 32 - protein, 7 aa, Artificial Sequence
  Artificially synthesized peptide sequence
VFSANHR

SEQ ID NO: 33 - protein, 7 aa, Artificial Sequence
  Artificially synthesized peptide sequence
VQLPHPP

SEQ ID NO: 34 - protein, 7 aa, Artificial Sequence
  Artificially synthesized peptide sequence
VPSANHR

SEQ ID NO: 35 - protein, 7 aa, Artificial Sequence
  Artificially synthesized peptide sequence
VTATRSM

SEQ ID NO: 36 - protein, 7 aa, Artificial Sequence
  Artificially synthesized peptide sequence
YASPRDT

SEQ ID NO: 37 - protein, 7 aa, Artificial Sequence
  Artificially synthesized peptide sequence
YGPTRPS

SEQ ID NO: 38 - protein, 7 aa, Artificial Sequence
  Artificially synthesized peptide sequence
YNRDSNQ

SEQ ID NO: 39 - DNA, 80 bp, Artificial Sequence
  Artificially synthesized oligonucleotide primer
aatgatacggcgaccaccgagatctacactctttccctacacgacgctcttccgatctttattcgcaattcctttagtgg

SEQ ID NO: 40 - DNA, 84 bp, Artificial Sequence
  Artificially synthesized oligonucleotide primer
caagcagaagacggcatacgagatcgtgatgtgactggagttcagacgtgtgctcttccgatcgccctcatagttagcgtaacg

SEQ ID NO: 41 - DNA, 84 bp, Artificial Sequence
  Artificially synthesized oligonucleotide primer
caagcagaagacggcatacgagatacatcggtgactggagttcagacgtgtgctcttccgatcgccctcatagttagcgtaacg

SEQ ID NO: 42 - DNA, 84 bp, Artificial Sequence
  Artificially synthesized oligonucleotide primer
caagcagaagacggcatacgagatgcctaagtgactggagttcagacgtgtgctcttccgatcgccctcatagttagcgtaacg

SEQ ID NO: 43 - DNA, 84 bp, Artificial Sequence
  Artificially synthesized oligonucleotide primer
caagcagaagacggcatacgagattggtcagtgactggagttcagacgtgtgctcttccgatcgccctcatagttagcgtaacg

SEQ ID NO: 44 - DNA, 84 bp, Artificial Sequence
  Artificially synthesized oligonucleotide primer
caagcagaagacggcatacgagatcactgtgtgactggagttcagacgtgtgctcttccgatcgccctcatagttagcgtaacg

SEQ ID NO: 45 - DNA, 84 bp, Artificial Sequence
  Artificially synthesized oligonucleotide primer
caagcagaagacggcatacgagatattggcgtgactggagttcagacgtgtgctcttccgatcgccctcatagttagcgtaacg

SEQ ID NO: 46 - DNA, 84 bp, Artificial Sequence
  Artificially synthesized oligonucleotide primer
caagcagaagacggcatacgagatgatctggtgactggagttcagacgtgtgctcttccgatcgccctcatagttagcgtaacg

SEQ ID NO: 47 - DNA, 84 bp, Artificial Sequence
  Artificially synthesized oligonucleotide primer
caagcagaagacggcatacgagattcaagtgtgactggagttcagacgtgtgctcttccgatcgccctcatagttagcgtaacg

SEQ ID NO: 48 - DNA, 84 bp, Artificial Sequence
  Artificially synthesized oligonucleotide primer
caagcagaagacggcatacgagatctgatcgtgactggagttcagacgtgtgctcttccgatcgccctcatagttagcgtaacg

SEQ ID NO: 49 - DNA, 84 bp, Artificial Sequence
  Artificially synthesized oligonucleotide primer
caagcagaagacggcatacgagataagctagtgactggagttcagacgtgtgctcttccgatcgccctcatagttagcgtaacg

SEQ ID NO: 50 - DNA, 84 bp, Artificial Sequence
  Artificially synthesized oligonucleotide primer
caagcagaagacggcatacgagatgtagccgtgactggagttcagacgtgtgctcttccgatcgccctcatagttagcgtaacg

SEQ ID NO: 51 - DNA, 84 bp, Artificial Sequence
  Artificially synthesized oligonucleotide primer
caagcagaagacggcatacgagattacaaggtgactggagttcagacgtgtgctcttccgatcgccctcatagttagcgtaacg

SEQ ID NO: 52 - DNA, 86 bp, Artificial Sequence
  Artificially synthesized oligonucleotide primer
caagcagaagacggcatacgagattgttgactgtgactggagttcagacgtgtgctcttccgatcgccctcatagttagcgtaacg

SEQ ID NO: 53 - DNA, 86 bp, Artificial Sequence
  Artificially synthesized oligonucleotide primer
caagcagaagacggcatacgagatacggaactgtgactggagttcagacgtgtgctcttccgatcgccctcatagttagcgtaacg

SEQ ID NO: 54 - DNA, 86 bp, Artificial Sequence
  Artificially synthesized oligonucleotide primer
caagcagaagacggcatacgagattctgacatgtgactggagttcagacgtgtgctcttccgatcgccctcatagttagcgtaacg

SEQ ID NO: 55 - DNA, 86 bp, Artificial Sequence
  Artificially synthesized oligonucleotide primer
caagcagaagacggcatacgagatcgggacgggtgactggagttcagacgtgtgctcttccgatcgccctcatagttagcgtaacg

SEQ ID NO: 56 - DNA, 86 bp, Artificial Sequence
  Artificially synthesized oligonucleotide primer
caagcagaagacggcatacgagatgtgcggacgtgactggagttcagacgtgtgctcttccgatcgccctcatagttagcgtaacg

SEQ ID NO: 57 - DNA, 86 bp, Artificial Sequence
  Artificially synthesized oligonucleotide primer
caagcagaagacggcatacgagatcgtttcacgtgactggagttcagacgtgtgctcttccgatcgccctcatagttagcgtaacg

SEQ ID NO: 58 - DNA, 86 bp, Artificial Sequence
  Artificially synthesized oligonucleotide primer
caagcagaagacggcatacgagataaggccacgtgactggagttcagacgtgtgctcttccgatcgccctcatagttagcgtaacg

SEQ ID NO: 59 - DNA, 86 bp, Artificial Sequence
  Artificially synthesized oligonucleotide primer
caagcagaagacggcatacgagattccgaaacgtgactggagttcagacgtgtgctcttccgatcgccctcatagttagcgtaacg

SEQ ID NO: 60 - DNA, 86 bp, Artificial Sequence
  Artificially synthesized oligonucleotide primer
caagcagaagacggcatacgagattacgtacggtgactggagttcagacgtgtgctcttccgatcgccctcatagttagcgtaacg

SEQ ID NO: 61 - DNA, 86 bp, Artificial Sequence
  Artificially synthesized oligonucleotide primer
caagcagaagacggcatacgagatatccactcgtgactggagttcagacgtgtgctcttccgatcgccctcatagttagcgtaacg

SEQ ID NO: 62 - DNA, 86 bp, Artificial Sequence
  Artificially synthesized oligonucleotide primer
caagcagaagacggcatacgagatatatccaggtgactggagttcagacgtgtgctcttccgatcgccctcatagttagcgtaacg

SEQ ID NO: 63 - DNA, 86 bp, Artificial Sequence
  Artificially synthesized oligonucleotide primer
caagcagaagacggcatacgagataaaggaatgtgactggagttcagacgtgtgctcttccgatcgccctcatagttagcgtaacg

SEQ ID NO: 64 - protein, 12 aa, Artificial Sequence
  Artificially synthesized peptide
  MISC_FEATURE (11)..(11): The lysine at position 11 is optionally conjugated to a lfuorescent moiety, which can optionally be 5-carboxyfluorescein (5-FAM)
  MISC_FEATURE (11)..(11): The lysine at position 11 is optionally conjugated to a fluorescent moiety, which can optionally be 5-carboxyfluorescein (5-FAM)
MDLSLKPGGSKC

SEQ ID NO: 65 - protein, 12 aa, Artificial Sequence
  Artificially synthesized peptide
  MISC_FEATURE (11)..(11): The lysine at position 11 is optionally conjugated to a fluorescent moiety, optionally 5-carboxyfluorescein (5-FAM)
  MISC_FEATURE (11)..(11): The lysine at position 11 is optionally conjugated to a fluorescent moiety, optionally 5-carboxyfluorescein (5-FAM)
MNSIAIPGGSKC